SEQUENCE LISTING (2 sequences)

SEQ ID NO: 1 - protein, 257 aa, Homo sapiens
MSVLTPLLLRGLTGSARRLPVPRAKIHMNENLFSAFIAPTILGLPAAVLIILFPPLLIPTSKYLINNRLITTQQWLIKLTSKQMMTMHNTKGRTWSLMLVSLIIFIATTNLLGLLPHSFTPTTQLSMNLAMAIPLWAGTVIMGFRSKIKNALAHFLPQGTPTPLIPMLVIIETISLLIQPMALAVRLTANITAGHLLMHLIGSATLAMSTINLPSTLIIFTILILLTILEIAVALIQAYVFTLLVSLYLLDYKDDDK

SEQ ID NO: 2 - protein, 294 aa, Homo sapiens
MQTAGALFISPALIRCCTRGLIRPVSASFLNSPVNSSKQPSYSNFPLQVARREFQTSVVSRDIDTANLFASFIAPTILGLPAAVLIILFPPLLIPTSKYLINNRLITTQQWLIKLTSKQMMTMHNTKGRTWSLMLVSLIIFIATTNLLGLLPHSFTPTTQLSMNLAMAIPLWAGTVIMGFRSKIKNALAHFLPQGTPTPLIPMLVIIETISLLIQPMALAVRLTANITAGHLLMHLIGSATLAMSTINLPSTLIIFTILILLTILEIAVALIQAYVFTLLVSLYLLDYKDDDDK

(19) United States
(12) Patent Application Publication
(10) Pub. No.: US 2004/0072774 A1
(43) Pub. Date: Apr. 15, 2004

(54) Methods for expressing and targeting mitochondrial-DNA-encoded peptides and uses thereof

(76) Inventors: Giovanni Manfredi, New York, NY (US); Eric A. Schon, Bronx, NY (US)

Correspondence Address: Lina Genovesi, Ph.D., Esq., Brown Raysman Millstein Felder & Steiner LLP, 163 Madison Avenue, P. O. Box 1989, Morristown, NJ, 07962-1989, US

(21) Appl. No.: 10/371,592

(22) Filed: Feb. 21, 2003

Related U.S. Application Data
(60) Provisional application No. 60/358,935, filed on Feb. 23, 2002.

(51) Int. Cl. A61K 48/00; C12N 15/85; C12N 1/00

(52) U.S. Cl. 514/44; 435/455; 435/317.1

(57) ABSTRACT

The present invention provides methods for introducing functional peptides into organelles. Additionally, the present invention provides a method for correcting a phenotypic deficiency in a mammal that results from a mutation in the mammal's mitochondrial DNA (mtDNA). The present invention further provides a method for treating a mitochondrial disorder in a subject in need of treatment therefor. Also provided is an expression vector that is useful for introducing a functional peptide encoded by an mtDNA sequence into a mitochondrion. The present invention also provides eukaryotic cells transformed by expression vectors that are useful for introducing functional peptides into organelles. Finally, the present invention provides a pharmaceutical composition comprising a non-nuclear nucleic acid sequence encoding a peptide for introduction into an organelle, a nucleic acid sequence encoding an organelle-targeting signal, and a pharmaceutically-acceptable carrier.

### RELATED APPLICATION

This application claims the benefit of U.S. Provisional Application No. 60/358,935, filed Feb. 22, 2002.

### STATEMENT OF GOVERNMENT INTEREST

This invention was made with government support under NIH Grant Nos. HD 32062, EY12335, EY11123, NS36302, and NS28828. As such, the United States government has certain rights in this invention.

### BACKGROUND OF THE INVENTION

Mitochondria are subcellular organelles found in eukaryotic cells. Under normal conditions, most of a cell's energy needs are supplied by its mitochondria. Unlike most other subcellular organelles, mitochondria are semi-independent from the nucleus, and contain their own genetic material. Mitochondrial DNA (mtDNA) was discovered in 1963 (Nass and Nass, Intramitochondrial fibers with DNA characteristics. J. Cell Biol., 19:593-629, 1963), and, by 1981, human mtDNA had been fully sequenced (Anderson et al., Sequence and organization of the human mitochondrial genome. Nature, 290:457-65, 1981). In each mitochondrion, there may be 2-10 copies of mtDNA. mtDNA bears more resemblance to prokaryotic DNA than to eukaryotic DNA: (1) it is a double-stranded, circular DNA molecule; (2) the genes encoded by mtDNA do not have introns; and (3) it uses a genetic code that differs from the “universal” genetic code. Thirty-seven genes are encoded by mtDNA, 13 of them for coding peptides (http://www.neuro.wustl.edu/neuromuscular/mitosyn.htm. Mitochondrial disorders).

Mutations in mtDNA are fixed at a much higher rate (˜10-100 fold higher) than are mutations in nuclear DNA. Several factors contribute to this phenomenon. Firstly, while there are thousands of mtDNAs in a female germline cell, there is only one haploid nuclear genome. Secondly, mtDNA is frequently attacked by reactive oxygen species and other free radicals (Kirkinezos et al., Reactive oxygen species and mitochondrial diseases. Semin. Cell Dev. Biol., 12:449-57, 2001). Thirdly, while mitochondria have adequate DNA-repair systems for some types of mutations (e.g., “base-excision repair” pathway), they have inadequate or no DNA-repair systems for other types of mutations (e.g., “nucleotide excision repair” pathway). Over 100 pathogenic point mutations have been discovered in human mtDNA, as well as a number of mtDNA deletions and duplication mutations (DiMauro et al., Mutations in mtDNA: are the inventors scraping the bottom of the barrel? Brain Pathol., 10:431-41, 2000).

There are two categories of inheritable mitochondrial disorders: those of nuclear-DNA origin and those of mtDNA origin. Since most of the proteins in mitochondria are encoded by nuclear DNA, defects in mitochondrial-protein-encoding genes in the nucleus affect mitochondrial function. For example, an A341V point mutation in NDUFVI, which is encoded by nuclear DNA, can cause patients to develop myoclonic epilepsy (Schuelke et al., Mutant NDUFV I subunit of mitochondrial complex I causes leukodystrophy and myoclonic epilepsy. Nature Genet., 21:260-261, 1999). Nevertheless, a large number of mitochondrial diseases have been linked to mtDNA abnormalities, Many of these disorders are associated with tissues that have high energy expenditures, including brain, heart, and muscle tissue. Because the mitochondria from sperm are actively degraded after fertilization, all mtDNA is inherited from the egg (DiMauro et al., Mitochondrial encephalomyopathies: where next? http://www.malattiemetaboliche.it/articoli/mith.htm). Accordingly, although they can arise de novo, disorders induced by mtDNA abnormalities are more often inherited maternally.

The first mitochondrial disease was reported in 1962 by Luft et al., who described a patient with severe hypermetabolism, mild weakness, and normal thyroid function (Luft et al., A case of severe hypermetabolism of nonthyroid origin with a defect in the maintenance of mitochondrial respiratory control: A correlated clinical, biochemical, and morphological study. J. Clin. Invest., 41:1776-1804, 1962). Since then, a large number of other mitochondrial diseases have been identified.

For example, a G→A point mutation at nucleotide 11778 in the ND4 subunit gene of complex I was the first point mutation in the mitochondrial genome linked to a maternally-inherited human disease. It causes Leber hereditary optic neuropathy (LHON), a disorder that blinds patients during the second and third decades of life. Of all mitochondrial diseases, LHON is the most common (Chinnery et al., The epidemiology of pathogenic mitochondrial DNA mutations. Ann. Neurol., 48:188-93, 2000). Three mtDNA mutations (G3460A, G11778A, and T14484C) account for 95% of LHON cases, with the G11778A mutation being the most common, accounting for 50% of LHON cases (Chinnery et al., The epidemiology of pathogenic mitochondrial DNA mutations. Ann. Neurol., 48:188-93, 2000; Carelli et al., Biochemical features of mtDNA 14484 (ND6/M64V) point mutation associated with Leber's hereditary optic neuropathy. Ann. Neurol., 45:320-28, 1999). Each LHON mutation affects a different subunit of the nicotinamide adenine dinucleotide:ubiquinone oxidoreductase (complex I) in the oxidative phosphorylation pathway, where electrons first enter the electron transport chain (Wallace, D. C., Mitochondrial diseases in man and mouse. Science, 283:1482-88, 1999). This large enzyme consists of seven subunits (ND1, 2, 3, 4, 4L, 5, and 6) encoded by mtDNA; the remaining 35 subunits are nuclear encoded (Sazanov et al., Resolution of the membrane domain of bovine complex I into subcomplexes: implications for the structural organization of the enzyme. Biochemistry, 39:7229-35, 2000).

It is believed that mitochondrial oxidative phosphorylation deficiency due to mutations in complex I subunit genes plays a pivotal role in the development of LHON, although the precise pathophysiological events precipitating acute visual failure and cellular injury remain elusive. Each LHON mutation alters mtDNA-encoded intrinsic complex I membrane proteins; yet, surprisingly, the standard spectrophotometric assays of complex I activity in LHON cells containing the G11778A mutation in the ND4 subunit gene are reduced slightly (Vergani et al., MtDNA mutations associated with Leber's hereditary optic neuropathy: studies on cytoplasmic hybrid (cybrid) cells. Biochem. Biophys. Res. Commun., 210:880-88, 1995; Majander et al., Electron transfer properties of NADH:ubiquinone reductase in the ND1/3460 and the ND4/11778 mutations of the Leber hereditary optic neuroretinopathy (LHON). FEBS Lett., 292:289-92, 1991; Larsson et al., Leber's hereditary optic neuropathy and complex I deficiency in muscle. Ann. Neurol., 30:701-08, 1991; Brown et al., Functional analysis of lymphoblast and cybrid mitochondria containing the 3460, 11778, or 14484 Leber's hereditary optic neuropathy mitochondrial DNA mutation. J. Biol. Chem., 275:39831-836, 2000).

Only the G3460A mutation in the ND1 subunit gene reduces complex I activity markedly (Majander et al., Electron transfer properties of NADH:ubiquinone reductase in the ND1/3460 and the ND4/11778 mutations of the Leber hereditary optic neuroretinopathy (LHON). FEBS Lett., 292:289-92, 1991; Brown et al., Functional analysis of lymphoblast and cybrid mitochondria containing the 3460, 11778, or 14484 Leber's hereditary optic neuropathy mitochondrial DNA mutation. J. Biol. Chem., 275:39831-836, 2000; Cock et al., Functional consequences of the 3460-bp mitochondrial DNA mutation associated with Leber's hereditary optic neuropathy. J. Neurol. Sci., 165:10-17, 1999). However, clear evidence of complex I deficiency with all three pathogenic mutations comes from polarographic investigations, showing impairment of cellular respiration when driven by complex-I-linked substrates (Majander et al., Electron transfer properties of NADH:ubiquinone reductase in the ND1/3460 and the ND4/11778 mutations of the Leber hereditary optic neuroretinopathy (LHON). FEBS Lett., 292:289-92, 1991; Larsson et al., Leber's hereditary optic neuropathy and complex I deficiency in muscle. Ann. Neurol., 30:701-08, 1991; Brown et al., Functional analysis of lymphoblast and cybrid mitochondria containing the 3460, 11778, or 14484 Leber's hereditary optic neuropathy mitochondrial DNA mutation. J. Biol. Chem., 275:39831-836, 2000). It is unclear how these different degrees of changes in complex I function result in the same clinical picture of almost-simultaneous bilateral apoplectic visual failure during early adult life, but reductions in oxidative phosphorylation and cellular injury induced by reactive oxygen species are believed to be implicated (Esposito et al., Mitochondrial disease in mouse results in increased oxidative stress. Proc. Natl Acad. Sci. USA, 96:4820-25, 1999; Brown, M. D., The enigmatic relationship between mitochondrial dysfunction and Leber's hereditary optic neuropathy. J. Neurol. Sci., 165:1-5, 1999).

Unlike most other mitochondrial mutations that impair neurological and myocardial function and are often fatal, patients with LHON, though blind, have a normal life expectancy. Unfortunately, there is little propensity for spontaneous visual recovery in the G11778A LHON patients, and there is no effective therapy. One of many potential avenues for treatment is to utilize gene therapy to introduce a “normal” gene encoding the defective complex I subunit into the optic nerves of LHON patients. Although exogenous genes have been successfully imported into the nuclear genome to protect the optic nerve (Guy et al., Reporter expression persists 1 year after adeno-associated virus-mediated gene transfer to the optic nerve. Arch. Ophthalmol., 117:929-37, 1999; Guy et al., Adeno-associated viral-mediated catalase expression suppresses optic neuritis in experimental allergic encephalomyelitis. Proc. Natl Acad. Sci. USA, 95:13847-852, 1998), these methods cannot be applied directly to introduce genes into the mammalian mitochondrial genome.

Additionally, two mitochondrial disorders, NARP (neuropathy, ataxia, and retinitis pigmentosa) and MILS (maternally-inherited Leigh syndrome), are most commonly the results of a T→G point mutation at nucleotide 8993 of the ATPase 6 gene in human mtDNA (Holt et al., A new mitochondrial disease associated with mitochondrial DNA heteroplasmy. Am. J. Hum. Genet., 46:428-33, 1990). ATPase 6 is a subunit of complex V of the respiratory/oxidative phosphorylation system (F₀F₁-ATP synthase), which catalyzes the synthesis of ATP from ADP and inorganic phosphate. F₀F₁-ATP synthase is a membrane-associated polypeptide complex. The F₀ sector is embedded in the membrane, and functions as a proton channel; the F₁ sector projects into the inner membrane space, and performs the synthesis of ATP (Elston et al., Energy transduction in ATP synthase. Nature, 391:510-13, 1998; Noji et al., The rotary machine in the cell, ATP synthase. J. Biol. Chem., 276:1665-68, 2001). The F₀F₁-ATP synthase complex comprises at least 14 nuclear DNA-encoded subunits (α, β, γ, δ, ε, b, c, d, e, f, g, h, IF1, and OSCP) and 2 mtDNA-encoded subunits (ATPase 6 and ATPase 8). In cells and transmitochondrial cytoplasmic hybrids from NARP and MILS patients, who generally have the T8993G mutation in ATPase 6, ATP synthesis is reduced by approximately 50-70% (Garcia et al., Structure, functioning, and assembly of the ATP synthase in cells from patients with the T8993G mitochondrial DNA mutation. Comparison with the enzyme in Rho⁰ cells completely lacking mtDNA. J. Biol. Chem., 275:11075-81, 2000; Manfredi et al., Oligomycin induces a decrease in the cellular content of a pathogenic mutation in the human mitochondrial ATPase 6 gene. J. Biol. Chem., 274:9386-91, 1999; Tatuch and Robinson, The mitochondrial DNA mutation at 8993 associated with NARP slows the rate of ATP synthesis in isolated lymphoblast mitochondria. Biochem. Biophys. Res. Commun., 192:124-28, 1993; Vazquez-Memije et al., Comparative biochemical studies in fibroblasts from patients with different forms of Leigh syndrome. J. Inher. Metab. Dis., 19:43-50, 1996).

Like many other mtDNA point mutations, the T8993G mutation is recessive (Holt et al., A new mitochondrial disease associated with mitochondrial DNA heteroplasmy. Am. J. Hum. Genet., 46:428-33, 1990; de Vries et al., A second missense mutation in the mitochondrial ATPase6 gene in Leigh's syndrome. Ann. Neurol., 34:410-12, 1993). NARP patients, who usually survive into their 30s or 40s, have some combination of ataxia, dementia, developmental delay, proximal neurogenic muscle weakness, retinitis pigmentosa, seizures, and sensory neuropathy (Holt et al., A new mitochondrial disease associated with mitochondrial DNA heteroplasmy. Am. J. Hum. Genet., 46:428-33, 1990). Typically 70% of the mtDNA in the blood of asymptomatic or oligosymptomatic mothers of NARP patients has the T8993G mutation; the level of mtDNA having the T8993G mutation is raised to approximately 80-90% in NARP patients (White et al., Genetic counseling and prenatal diagnosis for the mitochondrial DNA mutations at nucleotide 8993. Am. J. Hum. Genet., 65:474-82, 1999). Infants who are born with a T8993G mutation exceeding 90-95% exhibit MILS, a rapidly-fatal encephalopathy (Tatuch et al., Heteroplasmic mtDNA mutation (T→G) at 8993 can cause Leigh's disease when the percentage of abnormal mtDNA is high. Am. J. Hum. Genet., 50:852-58, 1992). Currently, no treatment is available for LHON, NARP, MILS, or any other mitochondrial disorders, many of which are lethal.

Early research in yeast utilized an engineered nucleus-localized version of an mtDNA-encoded gene specifying a cytoplasmically-expressed polypeptide that could be imported into mitochondria (Law et al., Studies on the import into mitochondria of yeast ATP synthase subunits 8 and 9 encoded by artificial nuclear genes. FEBS Lett., 236:501-05, 1988). Although this approach has been established in yeast, it has not been previously applied successfully to mammalian systems (Owen et al., Recombinant adeno-associated virus vector-based gene transfer for defects in oxidative metabolism. Hum. Gene Ther., 11(15):2067-78, 2000). Accordingly, there exists a need to develop therapeutic options for rescuing the deficiencies in mitochondrial oxidative phosphorylation, the deficiencies in ATP synthesis, and the other deficiencies found in patients suffering from conditions associated with defects in mtDNA.

### SUMMARY OF THE INVENTION

The present invention is based upon the inventors' successful rescue of ATP synthesis in mitochondria of mammalian cells by allotopic expression of a recoded MTATP6 gene, and their successful rescue of the mitochondrial oxidative phosphorylation deficiency of LHON by allotopic expression of a recoded ND4 subunit gene. Accordingly, in one aspect, the present invention provides a method for introducing a functional peptide encoded by a non-nuclear nucleic acid sequence into an organelle by: (a) preparing a nucleic-acid construct comprising a non-nuclear nucleic acid sequence encoding the peptide and a nucleic acid sequence encoding an organelle-targeting signal; (b) introducing the nucleic-acid construct into a eukaryotic cell to produce a transformed cell, wherein the eukaryotic cell is derived from algae, an animal, a multicellular or other non-yeast fungus, or protozoa; and (c) expressing the nucleic-acid construct from the nucleus of the transformed cell.

The present invention also provides a method for introducing a functional peptide encoded by a mitochondrial DNA (mtDNA) sequence into an organelle by:

(a) preparing a nucleic-acid construct, wherein the construct comprises an mtDNA sequence encoding the peptide and a nucleic acid sequence encoding an organelle-targeting signal;

(b) introducing the nucleic-acid construct into a eukaryotic cell to produce a transformed cell, wherein the eukaryotic cell is derived from algae, an animal, a plant, a multicellular or other non-yeast fungus, or protozoa; and (c) expressing the nucleic-acid construct from the nucleus of the transformed cell.

Additionally, the present invention provides a method for correcting a phenotypic deficiency in a mammal that results from a mutation in a peptide-encoding sequence of the mammal's mitochondrial DNA (mtDNA) by: (a) identifying the peptide-encoding sequence of the mammal's mtDNA in which the mutation occurs; (b) preparing a nucleic-acid construct comprising the peptide-encoding sequence of mtDNA and a nucleic acid sequence encoding a mitochondrial-targeting signal, wherein the peptide-encoding sequence of mtDNA encodes a wild-type peptide; (c) introducing the nucleic-acid construct into a mammalian cell to produce a transformed cell; and (d) expressing the nucleic-acid construct from the nucleus of the transformed cell.

In another aspect, the present invention provides a method for treating a mitochondrial disorder in a subject in need of treatment therefor, by administering to the subject a mitochondrial-DNA-encoded (mtDNA-encoded) peptide in an amount effective to treat the mitochondrial disorder.

The present invention further provides an expression vector that is useful for introducing a functional peptide encoded by a mitochondrial DNA (mtDNA) sequence into a mitochondrion, comprising: (a) a nucleic acid sequence encoding ATPase 6 subunit of F₀F₁-ATP synthase or ND4 subunit of complex I, wherein the nucleic acid sequence is compatible with the universal genetic code; and (b) a nucleic acid sequence encoding a mitochondrial-targeting signal, wherein the organelle-targeting signal is selected from the group consisting of the N-terminal region of human cytochrome c oxidase subunit VIII, the N-terminal region of the P1 isoform of subunit c of human ATP synthase, and the N-terminal region of the aldehyde dehydrogenase targeting sequence.

Also provided is a eukaryotic cell transformed by an expression vector that is useful for introducing a functional peptide encoded by a non-nuclear nucleic acid sequence into an organelle, wherein the eukaryotic cell is derived from algae, an animal, a multicellular or other non-yeast fungus, or protozoa, and the expression vector comprises: (a) a non-nuclear nucleic acid sequence encoding the peptide, wherein the nucleic acid sequence is compatible with the universal genetic code; and (b) a nucleic acid sequence encoding an organelle-targeting signal.

The present invention also provides a eukaryotic cell transformed by an expression vector that is useful for introducing a functional peptide encoded by a mitochondrial DNA (mtDNA) sequence into an organelle, wherein the eukaryotic cell is derived from algae, an animal, a plant, a multicellular or other non-yeast fungus, or protozoa, and the expression vector comprises: (a) an mtDNA sequence encoding the peptide, wherein the mtDNA sequence is compatible with the universal genetic code; and (b) a nucleic acid sequence encoding an organelle-targeting signal.

Finally, the present invention provides a pharmaceutical composition, comprising: (a) a non-nuclear nucleic acid sequence encoding a peptide for introduction into an organelle, wherein the nucleic acid sequence is compatible with the universal genetic code; (b) a nucleic acid sequence encoding an organelle-targeting signal; and (c) a pharmaceutically-acceptable carrier.

Additional aspects of the present invention will be apparent in view of the description which follows.

### BRIEF DESCRIPTION OF THE FIGURES

FIG. 1 illustrates the nucleic-acid constructs used in the present invention. a: Map and amino-acid sequence of C8A6F. The 11 ‘non-universal’ codons in MTATP6 (Met=ATA or ATG; Trp=TGA) are shown in bold. The sequence from COX8 (C8), containing the mitochondrial-targeting signal (MTS) (lower case) and 2 amino acids of mature COX VIII (IH), is underlined at the N terminus. The C-terminal FLAG epitope tag (F) is also in lower case. (The inventors added an extra encoded leucine (underlined), just before the FLAG epitope tag, for ease of plasmid construction.) Leu-156 of ATPase 6 (A6), which is mutated in NARP/MILS, is boxed. Note that recombinant A6 (rA6) lacks the last four C-terminal amino acids (HDNT). b: Map and amino-acid sequence of P1A6F. The sequence from ATP5G1, specifying the 61 amino acids of the MTS of the P1 isoform of ATPc (canonical residues for two-step cleavage of the precursor in bold) and 5 amino acids of mature ATPc (DIDTA) (P1), was placed upstream of rA6, which lacks the first three N-terminal amino acids (MNE). In some constructs (pTR-UF12-P1A6F-GFP; see FIG. 2), the inventors added an extra proline residue between the C terminus of the P1 MTS and the N terminus of rA6 (DTAPNLF). The remainder of the sequence is the same as C8A6F. Dots denote omitted sequence.

FIG. 2 demonstrates subcellular localization of rA6F in human 293T cells. a: Transient transfection of C8A6F inserted into pEF-BOS (top panel), and visualization by indirect immunofluorescence using antibodies to COX II and FLAG (middle panel). b: Transient transfection of P1A6F inserted into pEF-BOS (top panel), and visualization by staining with MitoTracker Red and anti-FLAG antibodies (middle panel). c: Transient infection of a P1A6F/GFP bicistronic construct inserted into AAV vector pTR-UF12 (top panel), and visualization with MitoTracker Red and anti-FLAG antibodies (middle panel). For each transfection, a merged image is shown in the bottom panel.

FIG. 3 depicts importation of rA6F into mitochondria. a: In vitro importation into rat mitochondria of in vitro-transcribed and -translated C8A6F (left panel) and P1A6F (right panel) constructs inserted into T7 promoter-based bacterial expression vectors. The predicted number of amino acids in the MTS, the precursor polypeptides (P), and the presumed mature polypeptides (M), are shown. Below each map is a fluorogram of the [³⁵S]-Met-labelled polypeptide translated in vitro in the importation assay. In each case, a portion of the in vitro-translated precursor is inside the organelle, as it is resistant to digestion by proteinase K (prot K). Sizes of molecular-weight markers are indicated, as are the positions of the predicted unprocessed and mature, processed polypeptides. The dot (right panel) denotes a band of unknown identity, perhaps due to incorrect processing of P1A6F. b: Western blot of in vivo importation of P1A6F into mitochondria in human 293T cells transiently transfected with either control empty plasmid (C) or with pEF-BOS-P1A6F. The inventors detected immunoreactive bands with anti-FLAG antibodies. Note that the predicted sizes do not correspond exactly to those implied by the molecular-weight markers (at left). This discrepancy is common when visualizing extremely hydrophobic proteins, such as ATPase 6 (A6), in SDS-PAGE44. c: Native Western blot of untransfected 293T cells, or cells transfected with pBOS-IRES-P1A6F or with pTR-UF12-P1A6F. Detection with antibodies to FLAG (right panel) on a blot from one gel, and with antibodies to F₁-ATPase subunit-α (left panel) on a blot from a duplicate gel run in parallel, showed co-migrating immunoreactive bands corresponding to a complex of approximately 600 kD, suggesting that rA6F was assembled together with F₁-α in complex V. Molecular-weight markers are shown at left.

FIG. 4 illustrates RT-PCR of stably-transfected cybrids. a: Maps of pEF-BOS-IRES-P1A6F plasmid DNA and the processed P1A6F mRNA after splicing of the intron (IVS derived from pIRES1-neo^{r}). Forward (F) primer A6-F and backward (B) primers IRES-B and Neo-B (arrows), and the predicted sizes of the various RT-PCR products, are shown. b: RT-PCR products of isolated RNA from pEF-BOS-IRES-P1A6F stably-transfected (RNA) and mock-transfected mutated cybrids (mock), as compared with PCR products from pEF-BOS-IRES-P1A6F plasmid DNA (DNA). M=100-bp ladder marker (sizes at left)

FIG. 5 shows phenotypes of homoplasmic mutated (8993T→G) cybrids transfected with P1A6F constructs. a: The left panel demonstrates transfection with pEF-BOS-IRES-P1A6F after G418 selection and growth in galactose-oligomycin for 3 d, followed by recovery in glucose-containing medium for the indicated number of days. The recovery rate of cybrids transfected with P1A6F is compared with that of mock-transfected cells (average of three independent experiments±s.d.). The dotted line denotes the cell number achieved at recovery day 5 by a similarly-selected 100% wild-type (WT; 8993T) cybrid line. The right panel depicts measurements of ATP synthesis in digitonin-permeabilized 100% wild-type and 100% mutant (8993G) cybrids with succinate (black bars) or with malate/pyruvate (gray bars) as a substrate, following mock transfection with empty vector pCDNA3 or with P1A6F constructs in pEF-BOS-IRES. Values are denoted as a percentage of the value in mock-transfected 100% wild-type lines. For each cell line, the inventors show the sensitivity of ATP synthesis to oligomycin as a white bar, the height of which denotes the fraction of ATP synthesis that was sensitive to 10 ng ml⁻¹ oligomycin, relative to the ATP synthesis measured in the same cells assayed with malate/pyruvate in the absence of oligomycin. n denotes number of experiments; error bars denote s.d. from the mean. b: Measurement of ATP synthesis in 100% mutant cybrids infected with three different AAV vectors containing P1A6F, or in mock-infected cells (with empty pTR-UF12), as compared with the values in mock-transfected, wild-type cybrids. Values that showed statistically-significant differences (P<0.05, by unpaired Student's t-test), as compared with mock-transfected cells, are indicated with an asterisk.

FIG. 6 illustrates immunoblotting of the P1ND4FLAG construct in UF-11. A: The top diagram shows the nuclear-encoded ND4 in AAV vector UF-11. Cellular infection with this construct should result in the synthesis of a 52-kD polypeptide, the molecular weight of the ND4Flag (bottom diagram). B: A Western blot of ND4Flag-transfected G11778A cybrids (lanes 1-4) shows a 52-kD band, consistent with expression of the ND4Flag fusion polypeptide (lanes 2, 3); in contrast, the control (untransfected cells; lanes 5-8) shows no staining with the anti-FLAG antibody. The stained gel shows the corresponding protein loading with successive 1-log unit dilutions (bottom half of gel). Overloading of lane 1 by cellular protein is readily apparent by the absence of any discrete pattern of protein bands in the stained gel. This contrasts with lane 2, in which discrete bands are best seen and the intensity of anti-Flag immunostaining is optimized. CMV=cytomegalovirus; TR=terminal repeat; CBA=chicken β-actin

FIG. 7 depicts immunocytochemistry of G11778A Leber hereditary optic neuropathy cybrids, including the cellular localization of mitochondria visualized by MitoTracker Red (A-C), FLAG visualized by indirect immunofluorescence using antibodies to FLAG (D-F) or to green fluorescent protein (GFP; G-I), and the merged images (J-L). Cells were transfected with P1ND4Flag inserted into the UF-11 adeno-associated virus (AAV) vector (column 1), the parent UF-11 vector (with no mitochondrial targeting sequence [MTS]; column 2), or AldhND4GFP inserted into UF-5 (column 3). Indicative of mitochondrial import, cells transfected with P1ND4Flag show that mitochondrially-targeted FLAG co-localizes with MitoTracker Red (J). In contrast, cells mock-transfected with the same AAV vector driving GFP expression in the place of the P1ND4Flag gene, and lacking a mitochondrial-targeting sequence, exhibit diffuse cytoplasmic staining of GFP only (H). This was not imported into mitochondria (K). Another construct, ND4 linked to GFP with the aldehyde dehydrogenase (Aldh) MTS, exhibited a punctate staining pattern (I). The relatively poor co-localization of GFP with MitoTracker Red (L) suggested that this ND4GFP fusion protein was not imported. Maps of the constructs used are shown below the micrographs.

FIG. 8 sets forth bar graphs of Leber hereditary optic neuropathy cybrid cell growth in selective medium, a complex I assay, and a complex V assay. A: Cell survival, after 3 days of media selection, of G11778A cybrids and wild-type cells transfected with P1ND4Flag, as compared with the mock-transfected cells (mean±standard deviation [SD]; n =10). B: Bar graph showing complex I (+III) activity in whole lysed cells. Results are expressed as the total cellular complex I activity, minus the value obtained after the addition of the complex I inhibitor, rotenone, giving the mitochondrial component of complex I activity (mean±SD; n=3). C: Bar graph showing the rate of ATP synthesis in permeabilized cells with pyruvate and malate serving as electron donors. Results represent total ATP levels detected in a luciferin-luciferase assay, in the presence of oligomycin—an inhibitor of the mitochondrial ATP synthase (mean±SD). Wt=wild-type; LHON=Leber hereditary optic neuropathy

### DETAILED DESCRIPTION OF THE INVENTION

The present invention provides a method for introducing a peptide encoded by a nucleic acid sequence into an organelle. Unless otherwise indicated, “peptide” shall include a protein, protein domain, polypeptide, peptide, or amino acid sequence, including any post-translational modification(s). One of skill in the art, upon reading the instant specification, will appreciate that these terms also include structural analogs and derivatives, e.g., peptides having conservative amino acid insertions, deletions, or substitutions, peptidomimetics, and the like. As further used herein, the term “organelle” refers to as membrane-bound structure that compartmentalizes functions within a eukaryotic cell, but does not include the nucleus of a cell. Examples of organelles that may be useful in the present invention include, without limitation, the endoplasmic reticulum, the Golgi complex (or Golgi apparatus), lysosomes (including primary and secondary lysosomes), mitochondria, plastids (including amyloplasts, chloroplasts, and chromoplasts), peroxisomes, ribosomes, secretory vesicles, and vacuoles.

In particular, the present invention provides a method for introducing a functional peptide encoded by a non-nuclear nucleic acid sequence into an organelle. As used herein, the term “functional peptide” refers to a peptide that demonstrates biological activity or function in a manner for which it was intended, and does not display a modification in its activity or functional properties as compared with the wild-type, or non-mutant, peptide. For example, where the peptide is an enzyme, or part of an enzyme complex, the peptide is functional if it demonstrates enzymatic activity (e.g., catalytic activity). The function of a peptide may be determined by standard assays that are well-known in the art, including those described herein. As further used herein, the term “non-nuclear” describes a nucleic acid sequence, or sequence of nucleotides (including DNA and RNA), that originates outside of the nucleus of a cell, and includes extrachromosomal DNA. For example, such a non-nuclear nucleic acid sequence could originate in an organelle (e.g., a chloroplast or a mitochondrion) or in the cytoplasm of the cell. In the method of the present invention, the peptide encoded by a non-nuclear nucleic acid sequence may be any peptide. In one embodiment of the present invention, the peptide is encoded by mitochondrial DNA (mtDNA). Examples of peptides encoded by mtDNA include, without limitation, ATPase 6 subunit of F₀F₁-ATP synthase, ATPase 8 subunit of F₀F₁-ATP synthase, and ND4 subunit of complex I. In a preferred embodiment of the present invention, the peptide is ATPase 6 subunit of F₀F₁-ATP synthase or ND4 subunit of complex I.

Additionally, in accordance with the method of the present invention, the organelle into which the peptide is introduced may be any of those described herein. Examples of such organelles include, without limitation, the endoplasmic reticulum, the Golgi complex (or Golgi apparatus), lysosomes (including primary and secondary lysosomes), mitochondria, plastids (including amyloplasts, chloroplasts, and chromoplasts), peroxisomes, ribosomes, secretory vesicles, and vacuoles. In one embodiment of the present invention, the organelle is a mitochondrion or a chloroplast. In a preferred embodiment of the present invention, the organelle is a mitochondrion.

The method of the present invention comprises the steps of: (a) preparing a nucleic-acid construct comprising a non-nuclear nucleic acid sequence encoding a peptide for introduction into an organelle and a nucleic acid sequence encoding an organelle-targeting signal; (b) introducing the nucleic-acid construct into a eukaryotic cell to produce a transformed cell, wherein the eukaryotic cell is derived from algae, an animal, a multicellular or other non-yeast fungus, or protozoa; and (c) expressing the nucleic-acid construct from the nucleus of the transformed cell. The functional peptide that is expressed may then be targeted to, and introduced into, the organelle under direction of the organelle-targeting signal.

In the method of the present invention, the non-nuclear nucleic acid sequence may be DNA or RNA (e.g., mitochondrial DNA or RNA), including synthetic forms and mixed polymers, and both sense and antisense strands. Nucleic acid sequences for use in the method of the present invention may be isolated from cell cultures using known methods. Additionally, the nucleic acid sequences may be prepared by a variety of techniques known to those skilled in the art, including, without limitation, the following: restriction enzyme digestion of nucleic acid; and automated synthesis of oligonucleotides, using commercially-available oligonucleotide synthesizers, such as the Applied Biosystems Model 392 DNA/RNA synthesizer. Furthermore, the non-nuclear nucleic acid sequence of the present invention may be derived from the same species as, or a different species from, that from which the eukaryotic cell of the present invention is derived.

An “organelle-targeting signal”, as used herein, is a peptide sequence, encoded by a nucleic acid sequence, that directs a peptide to its target organelle, including any organelle described herein. Many genes originally present in mitochondria and chloroplasts have been relocated, through time, to nuclear genomes. The products of their expression are targeted back to the appropriate organelles under the direction of organelle-targeting signals or transit peptides. Accordingly, the organelle-targeting signal of the present invention may be a peptide sequence that occurs in nature, which is added to a nuclear-DNA-encoded peptide that is generally transported to a target organelle. Additionally, the organelle-targeting signal of the present invention may be an artificial, or synthetic, peptide sequence which may correspond to a naturally-occurring transit sequence.

Examples of organelle-targeting signals that may be useful in the method of the present invention include, without limitation, the 32-amino-acid chloroplast transit sequence of the ribulose bisphosphatase carboxylase/oxygenase activase preprotein from Chlamydomonas reinhardtii; the N-terminal amphipathic alpha-helices of viral and cellular proteins; the N-terminal region of human cytochrome c oxidase subunit VIII; the primary sequence of the amino terminus of the Arabidopsis biotin synthase; the N-terminal region of the P1 isoform of subunit c of human ATP synthase; the N-terminal region of the aldehyde dehydrogenase targeting sequences; proteins that comprise the tetratricopeptide repeat (TPR), having four copies of the 34-amino-acid TPR motif; and other N-terminal hydrophilic or hydrophobic signal peptides. In one embodiment of the present invention, the organelle-targeting signal is the N-terminal region of human cytochrome c oxidase subunit VIII, the N-terminal region of the P1 isoform of subunit c of human ATP synthase, or the N-terminal region of the aldehyde dehydrogenase targeting sequence. Preferably, the organelle is a mitochondrion, the peptide is a mitochondrial-DNA-encoded (mtDNA-encoded) peptide, and the organelle-targeting signal is a mitochondrial targeting signal such as the N-terminal region of human cytochrome c oxidase subunit VIII, the N-terminal region of the P1 isoform of subunit c of human ATP synthase, or the N-terminal region of the aldehyde dehydrogenase targeting sequence.

In one preferred embodiment of the present invention, the mtDNA-encoded peptide is ATPase 6 subunit of F₀F₁-ATP synthase, and the organelle-targeting signal is the N-terminal region of human cytochrome c oxidase subunit VIII or the N-terminal region of the P1 isoform of subunit c of human ATP synthase. In another preferred embodiment, the mtDNA-encoded peptide is ND4 subunit of complex I, and the organelle-targeting signal is the N-terminal region of the P1 isoform of subunit c of human ATP synthase or the N-terminal region of the aldehyde dehydrogenase targeting sequence.

In accordance with the method of the present invention, a nucleic-acid construct may be prepared by methods known in the art, including those described below. Vectors, promoters, and ribosomal entry sites, including those disclosed herein, may be used, in conjunction with standard techniques, to prepare the nucleic-acid construct of the present invention. Vectors that may be useful in the present invention include, without limitation, bicistronic vectors (e.g., pEF-BOS-IRES), plasmid vectors, and adeno-associated virus (AAV) vectors (e.g., pTR-UF5, pTR-UF11, and pTR-UF12).

In the method of the present invention, the nucleic-acid construct may be labelled with a detectable marker, for facilitating detection of the peptide encoded within the nucleic-acid construct. Labelling may be accomplished using one of a variety of labelling techniques, including peroxidase, chemiluminescent labels known in the art, and radioactive labels known in the art. Additional detectable markers which may be useful in the method of the present invention include, without limitation, nonradioactive or fluorescent markers, such as biotin, fluorescein (FITC), acridine, cholesterol, and carboxy-X-rhodamine, which can be detected using fluorescence and other imaging techniques readily known in the art. Alternatively, the detectable marker may be a radioactive marker, including, for example, a radioisotope. The radioisotope may be any isotope that emits detectable radiation, such as ³⁵S, ³²P, or ³H. Radioactivity emitted by the radioisotope can be detected by techniques well known in the art. For example, gamma emission from the radioisotope may be detected using gamma imaging techniques, particularly scintigraphic imaging.

In accordance with the method of the present invention, the detectable marker may be encoded by a nucleic acid sequence that is incorporated within the nucleic-acid construct, resulting in expression of the detectable marker when the peptide of the present invention is expressed. Accordingly, in one embodiment of the present invention, the nucleic-acid construct further comprises a nucleic acid sequence encoding a detectable marker (e.g., an immunohistochemical marker). In a preferred embodiment of the present invention, the detectable marker is a FLAG epitope or green fluorescent protein (GFP). These markers then may be detected using anti-FLAG or anti-GFP antibodies in Western-blot analysis.

In the method of the present invention, the nucleic-acid construct is introduced into a eukaryotic cell, in a manner permitting expression of the peptide encoded by the non-nuclear nucleic acid sequence within the construct, thereby producing a transformed cell. The eukaryotic cell may be derived from algae, an animal, a multicellular or other non-yeast fungus, or protozoa. In one embodiment of the present invention, the eukaryotic cell is a mammalian cell, including a bone-marrow cell, a germ-line cell, a post-mitotic cell (e.g., a cell of the central nervous system), a progenitor cell, and a stem cell. In a preferred embodiment, the cell is a human cell, including a cell from a human cell line (e.g., human 293T HEK cells). In another preferred embodiment, the eukaryotic cell is a mammalian cell, the organelle is a mitochondrion, the peptide is a mitochondrial-DNA-encoded (mtDNA-encoded) peptide, and the organelle-targeting signal is the N-terminal region of human cytochrome c oxidase subunit VIII, the N-terminal region of the P1 isoform of subunit c of human ATP synthase, or the N-terminal region of the aldehyde dehydrogenase targeting sequence.

The nucleic-acid construct of the present invention may be introduced into the eukaryotic cell by standard methods of transfection or transformation known in the art. Examples of methods by which the construct may be introduced into the cell include, without limitation, electroporation, DEAE Dextran transfection, calcium phosphate transfection, cationic liposome fusion, protoplast fusion, creation of an in vivo electrical field, DNA-coated microprojectile bombardment, injection with a recombinant replication-defective virus, homologous recombination, ex vivo gene therapy, a viral vector, and naked DNA transfer, or any combination thereof. Recombinant viral vectors suitable for gene therapy include, but are not limited to, vectors derived from the genomes of viruses such as retrovirus, HSV, adenovirus, adeno-associated virus, Semiliki Forest virus, cytomegalovirus, and vaccinia virus.

It is within the confines of the present invention that the nucleic-acid construct may be introduced into the eukaryotic cell in vitro, using conventional procedures, to achieve expression in the cells of the peptide of the present invention. Eukaryotic cells expressing the peptide then may be introduced into a mammal, to provide the mammal with cells such that the functional peptide is expressed within the target organelle in vivo. In such an ex vivo gene therapy approach, the eukaryotic cells are preferably removed from the mammal, subjected to DNA techniques to incorporate the nucleic-acid construct, and then reintroduced into the mammal. However, the eukaryotic cells also may be derived from an organism other than the mammal, either of the same, or a different, species.

In the method of the present invention, the nucleic-acid construct is expressed from the nucleus of the eukaryotic cell into which it has been introduced. As used herein, the term “expressed from the nucleus” means that the transcription machinery of the nucleus, rather than the transcription machinery of an organelle, is used to generate an mRNA transcript of the peptide encoded by the non-nuclear nucleic acid sequence. Thereafter, the mRNA transcript is shuttled to the cytoplasm of the eukaryotic cell, wherein the transcript is translated into a functional peptide. The transcription-translation mechanisms of organelles are not involved. As disclosed herein, the nucleic acid sequence encoding the organelle-targeting signal is transcribed and translated along with the non-nuclear nucleic acid sequence encoding the peptide, such that the expressed peptide bears the organelle-targeting signal. It is this signal that then directs the expressed peptide in the cytoplasm of the eukaryotic cell to its targeted organelle. Expression of the peptide may be detected in the eukaryotic cell by detection methods readily determined from the known art, including, without limitation, immunological techniques (e.g., binding studies and Western blotting), hybridization analysis (e.g., using nucleic acid probes), fluorescence imaging techniques, and/or radiation detection. Similarly, the eukaryotic cell may be assayed, using standard protein assays known in the art or disclosed herein, for peptide function.

The method of the present invention may further comprise the step of mutagenizing the non-nuclear nucleic acid sequence encoding the peptide, if necessary, before step (a), to render the non-nuclear nulcleic acid sequence compatible with the universal genetic code, so as to permit “allotopic expression” of the non-nuclear nucleic acid sequence. Techniques for mutagenizing nucleic acids are well-known in the art (Herlitze and Koenen, A general and rapid mutagenesis method using polymerase chain reaction. Gene, 91:143-47, 1990; Sutherland et al., Multisite oligonucleotide-mediated mutagenesis: application to the conversion of a mitochondrial gene to universal genetic code. Biotechniques, 18:458-64, 1995).

All organisms that have been studied to date, including both prokaryotes and eukaryotes, generally use the same code for synthesis of proteins by cytoplasmic ribosomes. The exception to this occurs in mitochondria. Like chloroplasts in plants, mitochondria contain their own genetic information, and are capable of carrying out both transcription and translation. The genetic system of mitochondria differs from other known genetic systems because it deviates from the standard, or “universal”, genetic code in several ways. In particular, the UGA codon, which generally means “stop”, codes for tryptophan in mammalian mitochondria; the AUA codon, which generally codes for isoleucine, codes for methionine in mammalian mitochondria; and the AGA codon, which generally codes for arginine, means “stop” in mammalian mitochondria. Accordingly, where a mitochondrial nucleic acid sequence is used in the method of the present invention, it may be necessary to first mutagenize the nucleic acid sequence to render it compatible with the universal genetic code. In such instances, a mutagenized mtDNA-specified polypeptide is appended to a mitochondrial-targeting signal, expressed from the nucleus, and transported back to the mitochondria under the guidance of the signal peptide.

As described above, the method of the present invention may be used to introduce a peptide into an organelle in vitro, or in vivo in a mammal, by introducing the nucleic-acid construct of the present invention into a sufficient number of cells of the mammal (either in situ or initially ex vivo), in a manner permitting expression of the peptide encoded by the non-nuclear nucleic acid sequence contained within the construct. In view of the foregoing, the transformed eukaryotic cell of the present invention may be in, or introduced into, a mammal. The mammal may be any mammalian animal (e.g., humans, domestic animals, and commercial animals), but is preferably a human. Where the eukaryotic cell is already in a human, the organelle may be contained within any cell of the human, including bone-marrow cells, germ-line cells, post-mitotic cells (e.g., cells of the central nervous system), progenitor cells, and stem cells. Where ex vivo gene therapy techniques are used, the organelle may be initially contained within a eukaryotic cell (including a bone-marrow, germ-line, post-mitotic, progenitor, or stem cell) outside of the human, wherein the cell is preferably from the same species as the human, and, more preferably, from the human target. The eukaryotic cell containing the functional peptide within the targeted organelle then may be introduced into the human to permit in vivo proliferation of cells containing the functional peptide within the targeted organelles. The eukaryotic cell may be introduced into the human by standard techniques known in the art, including injection and transfusion.

In accordance with the present invention, the use of allotopic expression to introduce a functional peptide encoded by a non-nuclear nucleic acid sequence into an organelle may be utilized to rescue a mitochondrial disorder, such as a deficiency in ATP synthesis resulting from a defect in the mtDNA gene, MTATP6, or a mitochondrial oxidative phosphorylation deficiency resulting from a defect in the mtDNA gene, ND4 (a subunit of complex I). Without being bound by theory, it is believed that, by providing a method for introducing functional peptides into mitochondria, the allotopic-expression method of the present invention will be useful for the treatment of conditions associated with defects in mtDNA that result in defective peptides within the mitochondria. Accordingly, the method of the present invention may be particularly useful for treating mitochondrial disorders. Thus, the present invention provides a method for treating a mitochondrial disorder in a human in need of treatment, comprising introducing to the human the nucleic-acid construct of the present invention.

As used herein, a “mitochondrial disorder” is a condition, disease, or disorder characterized by a defect in activity or function of mitochondria, particularly a defect in mitochondrial activity or function that results from, or is associated with, a mutation in mtDNA. Examples of mitochondrial disorders include, without limitation, aging; aminoglycoside-induced deafness; cardiomyopathy; CPEO (chronic progressive external ophthalmoplegia); encephalomyopathy; FBSN (familial bilateral striatal necrosis); KS (Kearns-Sayre) syndrome; LHON (Leber hereditary optic neuropathy); MELAS (mitochondrial myopathy, encephalopathy, lactic acidosis, and stroke-like episodes); MERRF (myoclonic epilepsy with stroke-like episodes); MILS (maternally-inherited Leigh syndrome); mitochondrial myopathy; NARP (neuropathy, ataxia, and retinitis pigmentosa); PEO; SNE (subacute necrotizing encephalopathy). In one embodiment of the present invention, the mitochondrial disorder is associated with a mutation (e.g., a point mutation) in mtDNA. In another embodiment of the present invention, the mitochondrial disorder in the human is FBSN, MILS, or NARP, and the eukaryotic cell of the present invention (either in, or introduced into, the human) is transformed with a nucleic-acid construct comprising a non-nuclear nucleic acid sequence (e.g., mtDNA) that encodes wild-type ATPase 6 subunit of F₀F₁-ATP synthase. In a further embodiment of the present invention, the mitochondrial disorder in the human is LHON, and the eukaryotic cell of the present invention (either in, or introduced into, the human) is transformed with a nucleic-acid construct comprising a non-nuclear nucleic acid sequence (e.g., mtDNA) that encodes wild-type ND4 subunit of complex I.

A “mutation”, as used herein, is a permanent, transmissable change in genetic material. As further used herein, the term “wild-type” refers to the characteristic genotype (or phenotype) for a particular gene (or its gene product), as found most frequently in its natural source (e.g., in a natural population). A wild-type animal, for example, expresses functional ATPase 6 subunit of F₀F₁-ATP synthase or functional ND4 subunit of complex I.

The present invention also provides a method for introducing a functional peptide (as that term is described above) encoded by a mitochondrial DNA (mtDNA) sequence into an organelle. Examples of peptides encoded by mtDNA include, without limitation, ATPase 6 subunit of F₀F₁-ATP synthase, ATPase 8 subunit of F₀F₁-ATP synthase, and ND4 subunit of complex I. In a preferred embodiment of the present invention, the peptide is ATPase 6 subunit of F₀F₁-ATP synthase or ND4 subunit of complex I. The organelle into which the peptide is introduced may be any of those described herein. Examples of such organelles include, without limitation, the endoplasmic reticulum, the Golgi complex (or Golgi apparatus), lysosomes (including primary and secondary lysosomes), mitochondria, plastids (including amyloplasts, chloroplasts, and chromoplasts), peroxisomes, ribosomes, secretory vesicles, and vacuoles. In one embodiment of the present invention, the organelle is a mitochondrion or a chloroplast. In a preferred embodiment of the present invention, the organelle is a mitochondrion

The method of the present invention comprises the steps of: (a) preparing a nucleic-acid construct comprising an mtDNA sequence encoding a peptide for introduction into an organelle and a nucleic acid sequence encoding an organelle-targeting signal; (b) introducing the nucleic-acid construct into a eukaryotic cell to produce a transformed cell, wherein the eukaryotic cell is derived from algae, an animal, a multicellular or other non-yeast fungus, a plant, or protozoa; and (c) expressing the nucleic-acid construct from the nucleus of the transformed cell. The functional peptide that is expressed may then be targeted to, and introduced into, the organelle under direction of the organelle-targeting signal. The method of the present invention may further comprise the step of mutagenizing the mtDNA sequence encoding the peptide, before step (a), to render the mtDNA sequence compatible with the universal genetic code.

The present invention is also directed to a method for correcting a phenotypic deficiency in a mammal that results from a mutation in a peptide-encoding sequence of the mammal's mitochondrial DNA (mtDNA). As used herein, the term “phenotypic deficiency” refers to a defect in a mammal that manifests, at a cellular level, as subnormal activity or function of one or more peptides in the mammal, and can result in a condition, disease, or disorder in the mammal. In the method of the present invention, the phenotypic deficiency is caused by a mutation in mtDNA. By way of example, where a human has a mutation in the MTATP6 gene, ATP synthesis at the cellular level may be below the level normally expected in a healthy human, resulting in a mitochondrial disorder, such as FBSN, MILS, or NARP. As further used herein, the term “correcting a phenotypic deficiency” means rescuing or minimizing the deficiency by restoring, or partially restoring, at the cellular level, the activity or function of the defective peptide, thereby treating the condition, disease, or disorder in the mammal. Where, for example, a human suffers from a mitochondrial disorder such as FBSN, MILS, or NARP, as a result of a mutation in the MTATP6 gene, the phenotypic deficiency of defective ATP synthesis may be corrected by restoring, or partially restoring, activity or function of ATPase 6, thereby treating the mitochondrial disorder.

Accordingly, the present invention comprises a method for: (a) identifying the peptide-encoding sequence of the mammal's mtDNA in which the mutation occurs; (b) preparing a nucleic-acid construct comprising a peptide-encoding sequence of mtDNA and a nucleic acid sequence encoding a mitochondrial-targeting signal, wherein the peptide-encoding sequence of mtDNA encodes a wild-type peptide; (c) introducing the nucleic-acid construct into a mammalian cell to produce a transformed cell; and (d) expressing the nucleic-acid construct from the nucleus of the transformed cell. The functional peptide that is expressed in the cytosol of the cell may then be targeted to, and introduced into, mitochondria under direction of the mitochondrial-targeting signal. The method of the present invention may further comprise the step of mutagenizing the peptide-encoding sequence of mtDNA, before step (b), to render the mtDNA sequence compatible with the universal genetic code.

It is possible to identify the peptide-encoding sequence of the mammal's mtDNA in which the mutation of interest occurs by using standard techniques known in the art for isolating mtDNA, and for analyzing mtDNA to determine genetic defects. The mtDNA sequence of the present invention may be derived from the same species as, or a different species from, that from which the mammalian cell of the present invention is derived. Examples of peptide-encoding sequences of mtDNA for use in the present invention include, without limitation, mtDNA sequences that encode ATPase 6 subunit of F₀F₁-ATP synthase, ATPase 8 subunit of F₀F₁-ATP synthase, and ND4 subunit of complex I. In a preferred embodiment of the present invention, the peptide-encoding sequence of mtDNA encodes wild-type ATPase 6 subunit of F₀F₁-ATP synthase or wild-type ND4 subunit of complex I.

The present invention further provides a method for treating a mitochondrial disorder in a subject in need of treatment for a mitochondrial disorder. The mitochondrial disorder may be any of those described above. In one embodiment of the present invention, the mitochondrial disorder is associated with a mutation (e.g., a point mutation) in mtDNA. Preferably, the mitochondrial disorder is FBSN, LHON, MILS, or NARP. As used herein, the “subject” is a mammal, including, without limitation, a cow, dog, human, monkey, mouse, pig, or rat. Preferably, the subject is a human. The method of the present invention comprises administering to the subject a mitochondrial-DNA-encoded (mtDNA-encoded) peptide in an amount effective to treat the mitochondrial disorder. The mtDNA-encoded peptide may be any of those disclosed herein. In a preferred embodiment of the present invention, the peptide is ATPase 6 subunit of F₀F₁-ATP synthase or ND4 subunit of complex I. In another embodiment of the present invention, the mitochondrial disorder is FBSN, MILS, or NARP, and the mtDNA-encoded peptide is wild-type ATPase 6 subunit of F₀F₁-ATP synthase. In a further embodiment of the present invention, the mitochondrial disorder is LHON, and the mtDNA-encoded peptide is wild-type ND4 subunit of complex I.

The phrase “effective to treat the mitochondrial disorder”, as used herein, means effective to ameliorate or minimize the clinical impairment or symptoms resulting from the mitochondrial disorder. For example, where the subject suffers from NARP, the clinical impairment or symptoms of the disorder may be ameliorated or minimized by diminishing or alleviating ataxia, discomfort, neuropathy, pain, or retinitis pigmentosa experienced by the subject. The amount of peptide effective to treat a mitochondrial disorder in a subject in need of treatment therefor will vary depending on the particular factors of each case, including the type of mitochondrial disorder, the stage of the mitochondrial disorder, the subject's age and weight, the severity of the subject's condition, and the method of administration. These amounts can be readily determined by the skilled artisan, using techniques known in the art and/or disclosed herein.

In accordance with the method of the present invention, the mtDNA-encoded peptide may be administered to the subject by introducing into one or more cells of the subject an mtDNA sequence encoding the peptide, in a manner permitting expression of the peptide. Methods for carrying out this aspect of the present invention are described above. Without limitation, mtDNA encoding the peptide of the present invention may be introduced into the cells of the subject (either in situ in the subject or ex vivo) by standard methods of transfection or transformation known in the art, including electroporation, DEAE Dextran transfection, calcium phosphate transfection, cationic liposome fusion, protoplast fusion, creation of an in vivo electrical field, DNA-coated microprojectile bombardment, injection with a recombinant replication-defective virus, homologous recombination, ex vivo gene therapy, a viral vector, and naked DNA transfer, or any combination thereof. Recombinant viral vectors suitable for gene therapy include, but are not limited to, vectors derived from the genomes of viruses such as retrovirus, HSV, adenovirus, adeno-associated virus, Semiliki Forest virus, cytomegalovirus, and vaccinia virus.

Additionally, the mtDNA-encoded peptide may be administered to the subject by a method comprising the steps of: (a) obtaining a mtDNA sequence encoding the peptide; (b) mutagenizing the mtDNA sequence to render it compatible with the universal genetic code, thereby producing mutagenized mtDNA; (c) preparing a nucleic-acid construct comprising the mutagenized mtDNA and a nucleic acid sequence encoding a mitochondrial-targeting signal; (d) introducing the nucleic-acid construct into one or more cells of the subject; and (e) in at least one cell of the subject into which the nucleic-acid construct is introduced, expressing the nucleic-acid construct from the nucleus of the cell. The mtDNA-encoded peptide that is expressed in the cytosol of the cell may then be targeted to, and introduced into, the mitochondrion under direction of the mitochondrial-targeting signal. In one embodiment of the present invention, step (d) is performed ex vivo (outside of the subject).

Nucleic acid sequences for use in the method of the present invention may be isolated from cell cultures using known methods. Additional means for preparing the nucleic acid sequences have been described previously, and include, without limitation, the following: restriction enzyme digestion of nucleic acid; and automated synthesis of oligonucleotides, using commercially-available oligonucleotide synthesizers, such as the Applied Biosystems Model 392 DNA/RNA synthesizer. Furthermore, the mtDNA sequence of the present invention may be derived from the same species as, or a different species from, that from which the cells of the present invention are derived. Likewise, the mitochondrial-targeting signal which has been previously described may include a peptide sequence that occurs in nature, and which is added to an mtDNA-encoded peptide that is generally transported to a target organelle.

The present invention further provides an expression vector that is useful for introducing a functional peptide encoded by a mitochondrial DNA (mtDNA) sequence into a mitochondrion. The phrase “expression vector” generally refers to nucleotide sequences that are capable of effecting expression of a structural gene in hosts compatible with such sequences. These expression vectors typically include at least suitable promoter sequences and, optionally, termination signals. The selection of suitable promoter sequences is well known in the art, as is the selection of appropriate expression vectors. (See, e.g., Sambrook et al., Molecular Cloning: A Laboratory Manual (2d ed.), vols. 1-3, Cold Spring Harbor Laboratory, 1989.)

The expression vector of the present invention comprises a nucleic acid sequence encoding ATPase 6 subunit of F₀F₁-ATP synthase or ND4 subunit of complex I, wherein the nucleic acid sequence is compatible with the universal genetic code; and a nucleic acid sequence encoding a mitochondrial-targeting signal, wherein the mitochondrial-targeting signal is the N-terminal region of human cytochrome c oxidase subunit VIII, the N-terminal region of the P1 isoform of subunit c of human ATP synthase, or the N-terminal region of the aldehyde dehydrogenase targeting sequence. In one embodiment of the present invention, the mitochondrial-targeting signal is the N-terminal region of human cytochrome c oxidase subunit VIII or the N-terminal region of the P1 isoform of subunit c of human ATP synthase. In another embodiment, the mitochondrial-targeting signal is the N-terminal region of the P1 isoform of subunit c of human ATP synthase or the N-terminal region of the aldehyde dehydrogenase targeting sequence.

As previously discussed, the expression vector of the present invention may be prepared by methods known in the art, including those described below. Promoters and ribosomal entry sites, including those disclosed herein, may be used, in conjunction with standard techniques, to prepare the expression vector of the present invention. Vectors that may be useful in the present invention include, without limitation, bicistronic vectors (e.g., pEF-BOS-IRES), plasmid vectors, and adeno-associated virus (AAV) vectors (e.g., pTR-UF5, pTR-UF11, and pTR-UF12). Additionally, in accordance with the present invention, the expression vector may be labelled with a detectable marker, for facilitating detection of the ATPase 6 subunit of F₀F₁-ATP synthase. Labelling may be accomplished using one of a variety of labelling techniques, including any of those described herein. In a preferred embodiment of the present invention, the detectable marker is a FLAG epitope or GFP. This marker then may be detected using anti-FLAG or anti-GFP antibodies in Western-blot analysis.

Further provided in the present invention are eukaryotic cells transformed by the above-described expression vectors. The eukaryotic cells may be derived from algae, animals, plants, multicellular and other non-yeast fungi, or protozoa. The present invention also provides clonal cell strains comprising the transformed eukaryotic cells described herein.

The present invention further provides a eukaryotic cell transformed by an expression vector that is useful for introducing a functional peptide encoded by a non-nuclear nucleic acid sequence into an organelle, wherein the expression vector comprises: (a) a non-nuclear nucleic acid sequence encoding the peptide, wherein the nucleic acid sequence is compatible with the universal genetic code; and (b) a nucleic acid sequence encoding an organelle-targeting signal. The eukaryotic cell may derived from algae, an animal, a multicellular or other non-yeast fungus, or protozoa. Preferably, the cell is a mammalian cell. More preferably, the cell is a human cell (e.g., a bone-marrow cell; a clonal cell; a germ-line cell; a post-mitotic cell, such as a cell of the central nervous system; a progenitor cell; and a stem cell). For example, the human cell line, 293T HEK, is particularly useful in the practice of the present invention. In one embodiment of the present invention, the eukaryotic cell expresses the functional peptide. The present invention also provides clonal cell strains comprising the transformed eukaryotic cells described herein.

In the transformed eukaryotic cell of the present invention, the non-nuclear nucleic acid sequence encoding the peptide may be any non-nuclear sequence. In one embodiment of the present invention, the non-nuclear nucleic acid sequence is mitochondrial DNA (mtDNA). Examples of peptides encoded by mtDNA include, without limitation, ATPase 6 subunit of F₀F₁-ATP synthase, ATPase 8 subunit of F₀F₁-ATP synthase, and ND4 subunit of complex I. In a preferred embodiment of the present invention, the non-nuclear nucleic acid sequence encodes ATPase 6 subunit of F₀F₁-ATP synthase or ND4 subunit of complex I. As previously discussed in greater detail, the organelle-targeting signal of the present invention may be derived from a peptide sequence that occurs in nature, which is added to a nuclear-DNA-encoded peptide that is generally transported to a target organelle. Additionally, the organelle-targeting signal of the present invention may be an artificial or synthetic peptide sequence, which may correspond to a naturally-occurring transit sequence.

The present invention further provides a eukaryotic cell transformed by an expression vector that is useful for introducing a functional peptide encoded by a mitochondrial DNA (mtDNA) sequence into an organelle, wherein the expression vector comprises: (a) an mtDNA sequence encoding the peptide, wherein the mtDNA sequence is compatible with the universal genetic code; and (b) a nucleic acid sequence encoding an organelle-targeting signal. The eukaryotic cell may derived from algae, an animal, a multicellular or other non-yeast fungus, a plant, or protozoa. Preferably, the cell is a mammalian cell. More preferably, the cell is a human cell (e.g., a bone-marrow cell; a clonal cell; a germ-line cell; a post-mitotic cell, such as a cell of the central nervous system; a progenitor cell; and a stem cell). In one embodiment of the present invention, the eukaryotic cell expresses the functional peptide. The present invention also provides clonal cell strains comprising the transformed eukaryotic cells described herein.

The present invention is also directed to a pharmaceutical composition, comprising: (a) a non-nuclear nucleic acid sequence encoding a peptide for introduction into an organelle, wherein the nucleic acid sequence is compatible with the universal genetic code; (b) a nucleic acid sequence encoding an organelle-targeting signal; and (c) a pharmaceutically-acceptable carrier. Preferred non-nuclear nucleic acid sequences encoding the peptide for introduction into an organelle, as well as the organelle-targeting signal, have been described above.

The pharmaceutically-acceptable carrier must be “acceptable” in the sense of being compatible with the other ingredients of the composition, and not deleterious to the recipient thereof. Examples of acceptable pharmaceutical carriers include carboxymethyl cellulose, crystalline cellulose, glycerin, gum arabic, lactose, magnesium stearate, methyl cellulose, powders, saline, sodium alginate, sucrose, starch, talc, and water, among others. Formulations of the pharmaceutical composition may be conveniently presented in unit dosage.

The formulations of the present invention may be prepared by methods well-known in the pharmaceutical art. For example, the nucleic acid sequences may be brought into association with a carrier or diluent, as a suspension or solution. Optionally, one or more accessory ingredients (e.g., buffers, flavoring agents, surface active agents, and the like) also may be added. The choice of carrier will depend upon the route of administration. The pharmaceutical composition would be useful for administering the nucleic acid sequences of the present invention to a subject to treat a mitochondrial disorder. The mtDNA sequence encoding the peptide is provided in an amount that is effective to treat a mitochondrial disorder in the subject. That amount may be readily determined by the skilled artisan, as described above.

The present invention is further illustrated by the following examples, which are set forth to aid in the understanding of the invention, and should not be construed to limit in any way the scope of the invention as defined in the claims which follow thereafter.

### EXAMPLES

### Example 1

Preparation of Constructs

The inventors inserted C8A6F (comprising the sequence from COX8 (C8), containing the mitochondrial-targeting signal (MTS) and 2 amino acids of mature COX VIII, ATPase 6 (A6), and a C-terminal FLAG epitope tag (F)) and P1A6F (the sequence from ATP5G1, specifying the 61 amino acids of the MTS of the P1 isoform of ATPc and 5 amino acids of mature ATPc (P1), ATPase 6 (A6), and a C-terminal FLAG epitope tag (F)) into the XbaI sites of pEF-BOS (Mizushima and Nagata, pEF-BOS, a powerful mammalian expression vector. Nucleic Acids Res., 18:5322, 1990) and pEF-BOS-IRES—a bicistronic vector containing the promoter for eukaryotic translation factor EF1-α, derived from pEF-BOS, and an internal ribosomal entry site (IRES), derived from pIRES1-neo^{r} (Clontech) (Rees et al., Bicistronic vector for the creation of stable mammalian cell lines that predisposes all antibiotic-resistant cells to express recombinant protein. Biotechniques, 20:102-10, 1996). The inventors also inserted a P1A6F construct into the XbaI site of the adeno-associated virus (AAV) vectors, pTR-UF5 (regulated by a cytomegalovirus (CMV) immediate early promoter) (Zolotukhin et al., A “humanized” green fluorescent protein cDNA adapted for high-level expression in mammalian cells. J. Virol., 70:4646-54, 1996), and pTR-UF11 and pTR-UF12 (both regulated by the 381-bp CMV immediate early gene enhancer/1352-bp chicken β-actin (CBA) promoter-exonl-intronl). To generate mitochondrially-targeted expression of P1A6F and cytoplasmically-targeted expression of green fluorescent protein (GFP) in the same cell, the inventors used a pTR-UF12 construct that had P1A6F linked to GFP via a 637-bp poliovirus IRES. Visualization of GFP enabled the inventors to identify those cells that were probably expressing P1A6F (inserted upstream of the IRES). The inventors amplified the plasmids, purified them by cesium chloride gradient centrifugation, then packaged them into recombinant AAV (rAAV) by transfection into human 293 cells using standard procedures (Zolotukhin et al., A “humanized” green fluorescent protein cDNA adapted for high-level expression in mammalian cells. J. Virol., 70:4646-54, 1996). The rAAV was titered by an infectious center assay (Hauswirth et al., Production and purification of recombinant adeno-associated virus. Methods Enzymol., 316:743-61, 2000).

### Example 2

Cell Culture and Viral Transfection

The inventors cultured homoplasmic cybrids containing wild-type (8993T) and mutated (8993G) mtDNA, as described (Manfredi et al., Oligomycin induces a decrease in the cellular content of a pathogenic mutation in the human mitochondrial ATPase 6 gene. J. Biol. Chem., 274:9386-91, 1999). For AAV infections, the inventors infected cybrids at approximately 80% confluency with 3.0×10⁷ AAV or rAAV viral particles. The inventors selected in galactose-oligomycin in triplicate, as described (Manfredi et al., Oligomycin induces a decrease in the cellular content of a pathogenic mutation in the human mitochondrial ATPase 6 gene. J. Biol. Chem., 274:9386-91, 1999), except that the cells were treated with selective medium for just 3 d. Thereafter, the selective medium was replaced with complete high-glucose medium.

### Example 3

In Vitro Transcription, Translation, and Importation Assays

The inventors inserted P1A6F into the prokaryotic expression vector, pCR II (Invitrogen Corporation, Carlsbad, Calif.). The inventors carried out in vitro transcription and translation with an SP6 TNT Quick Coupled rabbit reticulocyte lysate system (Promega, Madison, Wis.) in the presence of [³⁵S]-Met, according to the manufacturer's protocol. For mitochondrial importation, the inventors isolated fresh rat liver mitochondria (Isaya et al., Sequence analysis of rat mitochondrial intermediate peptidase: similarity to zinc metallopeptidases and to a putative yeast homologue. Proc. Natl Acad. Sci. USA, 89:8317-21, 1992). For mitochondrial import assays (Isaya et al., Sequence analysis of rat mitochondrial intermediate peptidase: similarity to zinc metallopeptidases and to a putative yeast homologue. Proc. Natl Acad. Sci. USA, 89:8317-21, 1992), the inventors incubated each of two aliquots of 12 μl of radiolabelled translation mixture with 6 μl of permeabilized mitochondria, for 30 min at 27° C. The inventors treated one aliquot with 250 μg ml⁻¹ proteinase K for 30 min on ice, then added 1 mM PMSF. The inventors rinsed both aliquots in 40 μl HMS (2 mM HEPES/KOH, pH 7.4; 220 mM mannitol; 70 mM sucrose), and pelleted them, by centrifugation at 11,000 g, for 1 min. The inventors resuspended the pellets in Laemmli sample buffer, and electrophoresed them through a 10% SDS-PAGE gel. The inventors then dried the gels, and subjected them to autoradiography for 48 h. To determine whether importation was dependent upon mitochondrial membrane potential, the inventors pre-treated freshly-isolated rat liver mitochondria with 30 μM carbonyl cyanide p-trifluoromethoxy phenylhydrazone (FCCP), for 5 min on ice, before using it in the in vitro importation assay described above.

### Example 4

Immunological Techniques

For immunohistochemistry, the inventors transiently transfected human 293T HEK cells grown on glass slides with pEF-BOS-based constructs, using FuGENE6 Transfection Reagent (Roche), according to the manufacturer's protocol. Alternatively, the inventors infected 293T cells with AAV, as described above. After 48 h, the inventors incubated the cells for 30 min with 250 nM of the mitochondrial-specific fluorescent dye, MitoTracker Red (Molecular Probes). The inventors carried out immunostaining with mouse monoclonal anti-FLAG M2 antibodies (Sigma Immunochemicals), as described (Sciacco and Bonilla, Cytochemistry and immunocytochemistry of mitochondria in tissue sections. Methods Enzymol., 264:509-21, 1996). The inventors used secondary anti-mouse Cy5 or Cy2, and anti-rabbit Cy2 (Jackson Immunochemicals), for immunodetection, and visualized immunofluorescence in a Zeiss Confocal microscope. The inventors visualized selected digital images with different pseudocolors for MitoTracker, FLAG, or COX II, as appropriate, and merged them in RGB format for evaluation of co-localization.

For Western-blot analysis, the inventors transfected the 293T cells with pEF-BOS-P1A6F, as described above. The inventors electrophoresed 40 μg of proteins from total cellular lysates and from isolated mitochondria (Pallotti and Lenaz, Isolation and subfractionation of mitochondria from animal cells and tissue culture lines. Methods Cell Biol., 65:1-35, 2001), with and without pretreatment with 250 μg ml⁻¹ proteinase K (Isaya et al., Sequence analysis of rat mitochondrial intermediate peptidase: similarity to zinc metallopeptidases and to a putative yeast homologue. Proc. Natl Acad. Sci. USA, 89:8317-21, 1992), through a 15% polyacrylamide gel, then electro-transferred the proteins to a polyvinylidene fluoride membrane (Bio-Rad, Hercules, Calif.). The inventors immunostained the membrane with mouse monoclonal anti-FLAG M2 antibodies, and then with rabbit anti-mouse IgG HRP-conjugated secondary antibodies. The inventors detected proteins using a chemiluminescence system (Amersham Pharmacia), and quantified the immunostained fragments by densitometry, using a Fluor-S MultiImager System (Bio-Rad, Hercules, Calif.).

For native Western-blot analyses, the inventors prepared mitochondria-enriched fractions of 293T HEK cells transfected with pBOS-IRES-P1A6F or with pTR-UFi2-P1A6F, as described (Klement et al., Analysis of oxidative phosphorylation complexes in cultured human fibroblasts and amniocytes by blue-native-electrophoresis using mitoplasts isolated with the help of digitonin. Anal. Biochem., 231:21-24, 1995). The inventors solubilized mitochondria with mild detergents (Klement et al., Analysis of oxidative phosphorylation complexes in cultured human fibroblasts and amniocytes by blue-native-electrophoresis using mitoplasts isolated with the help of digitonin. Anal. Biochem., 231:21-24, 1995), and electrophoresed 10-20 μg of proteins, in duplicate, through a nonlinear 4-15% polyacrylamide gradient gel, under non-denaturing conditions. The inventors electro-transferred and immunodetected the proteins, as described above, using antibodies to FLAG M2 on one membrane, and to ATPase subunit α (Molecular Probes) on the other.

### Example 5

Polymearse Chain Reaction (PCR) and Reverse Transcriptase PCR (RT-PCR)

The inventors detected the 8993T→G mutation in cybrid cell lines by RFLP of PCR products with Aval, as previously described (Manfredi et al., Oligomycin induces a decrease in the cellular content of a pathogenic mutation in the human mitochondrial ATPase 6 gene. J. Biol. Chem., 274:9386-91, 1999). For RT-PCR, the inventors extracted total RNA from 8993T→G mutated cybrid cells mock-transfected or stably transfected with pBOS-IRES-P1A6F, using a Totally RNA extraction kit (Ambion). The inventors generated cDNA by reverse transcription of polyadenylated RNA with oligo(dT) primers, using a Thermoscript RT-PCR system (Gibco-BRL Life Technologies, Gaithersburg, Md.). The inventors amplified cDNA sequences and pBOS-IRES-P1A6F plasmid DNA by PCR.

### Example 6

ATP Synthesis

The inventors measured ATP synthesis in whole permeabilized cells using succinate or malate plus pyruvate as substrates, as described (Manfredi et al., Assay of mitochondrial ATP synthesis in animal cells. Methods Cell Biol., 65:133-45, 2001). The inventors also measured ATP synthesis with malate plus pyruvate, after the addition of 10 ng ml⁻¹ oligomycin, to test for sensitivity to low doses of a specific ATPase inhibitor.

Discussed below are results obtained by the inventors in connection with the experiments of Examples 1-6:

Allotopic Expression Strategy

To effect an allotopic expression strategy for MTATP6, two key obstacles need to be overcome. The first obstacle is the problem of the human mitochondrial genetic code, which differs from the nuclear universal code at 4 of the 64 codon positions. Simply transferring a cloned mitochondrial ATPase 6 gene to the nucleus will result in translation of a missense and/or truncated polypeptide. The second obstacle is the need to target this recoded ATPase 6 to mitochondria.

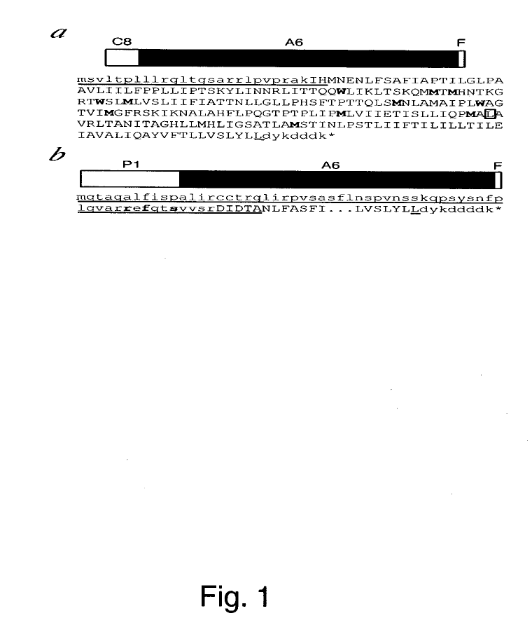

To overcome the first hurdle, the inventors recoded all 11 non-universal codons in MTATP6 (FIG. 1) by in vitro mutagenesis (Herlitze and Koenen, A general and rapid mutagenesis method using polymerase chain reaction. Gene, 91:143-47, 1990; Sutherland et al., Multisite oligonucleotide-mediated mutagenesis: application to the conversion of a mitochondrial gene to universal genetic code. Biotechniques, 18:458-64, 1995). To overcome the second hurdle, the inventors appended to the recoded ATPase 6 (rA6) gene sequences from COX8 specifying the N-terminal region of the nucleus-encoded and mitochondrially-targeted subunit VIII of human cytochrome c oxidase (C8), which contains the entire 25-amino-acid mitochondrial-targeting signal (MTS) plus the first 2 amino acids of the mature COX VIII polypeptide (C8A6F; FIG. 1a) (Rizzuto et al., A gene specifying subunit VIII of human cytochrome c oxidase is localized to chromosome 11 and is expressed in both muscle and non-muscle tissues. J. Biol. Chem., 264:10595-600, 1989; Rizzuto et al., Rapid changes of mitochondrial Ca2+ revealed by specifically targeted recombinant aequorin. Nature, 358:325-27, 1992). Because no suitable antibody to human ATPase 6 (A6) was available, the inventors appended a FLAG epitope tag (F) to the C terminus of the rA6 gene. The inventors also made constructs in which C8 was replaced by sequences from ATP5G1 specifying the N-terminal region of the P1 isoform (P1) of subunit c of human ATP synthase (ATPc) (Higuti et al., Molecular cloning and sequence of two cDNAs for human subunit c of H+-ATP synthase in mitochondria. Biochim. Biophys. Acta, 1173:87-90, 1993), which contains the entire 61-amino-acid MTS plus the first 5 amino acids of the mature P1 polypeptide (P1A6F; FIG. 1b). The inventors inserted versions of both constructs into plasmid and adeno-associated virus (AAV) vectors.

Allotopic Expression of Recoded MTATP6 in Normal Cells

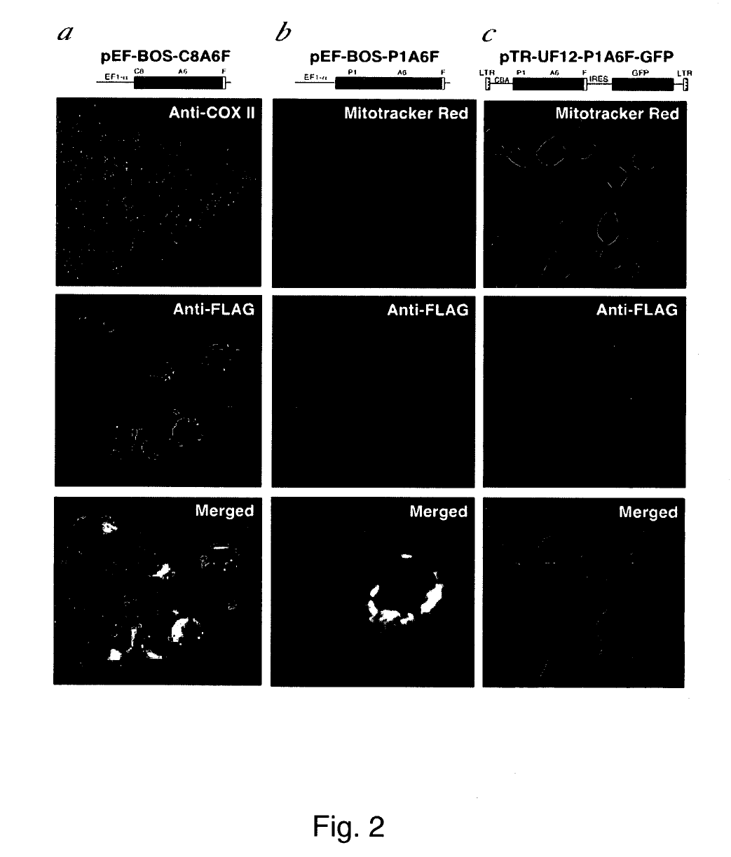

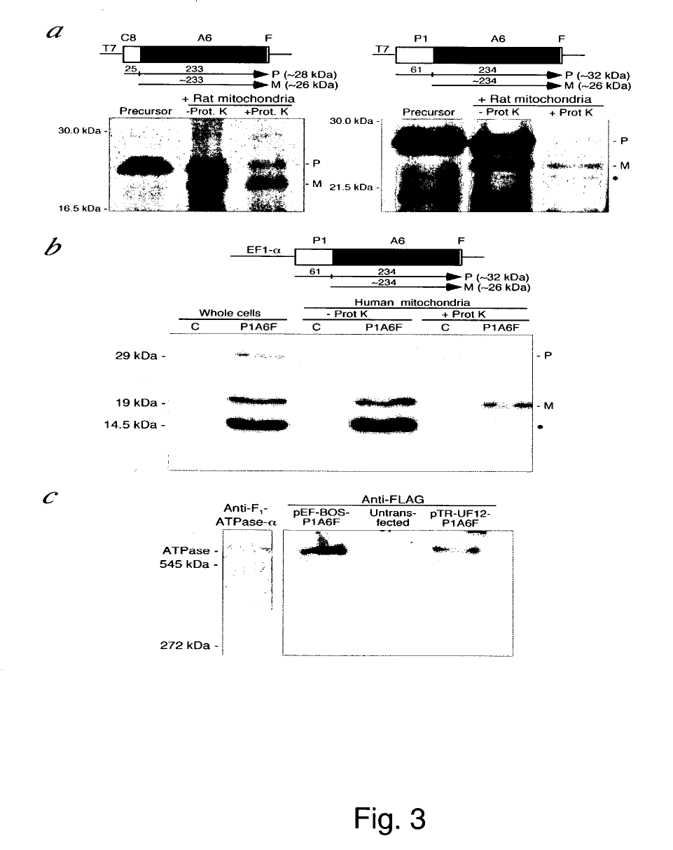

Transient expression of both C8A6F (FIG. 2a) and P1A6F (FIG. 2b, c) in human 293T HEK cells showed that both presequences were able to direct the allotopically-expressed polypeptide to mitochondria. Immunohistochemistry to detect the FLAG epitope not only showed a typical punctate mitochondrial pattern, but also co-localized with both subunit II of cytochrome c oxidase (COX II), an mtDNA-encoded subunit of complex IV of the respiratory chain (FIG. 2a), and the mitochondrion-specific dye, MitoTracker Red (FIG. 2b, c). In addition, using a coupled in vitro transcription-translation system to synthesize C8A6F and P1A6F precursors labelled with [³⁵S]-Met, the inventors determined that both the C8 and P1 mitochondrial-targeting signals were able to direct importation of the respective precursors into isolated rat liver mitochondria (FIG. 3a) (Ryan et al., Assaying protein import into mitochondria. Methods Cell Biol., 65:189-215, 2001). The imported polypeptides were resistant to proteinase K treatment of isolated mitochondria, and had sizes consistent with those of the respective mature polypeptides (i.e., with the MTS removed) (FIG. 3a). Since the MTS of P1 contains a canonical recognition sequence for two-step cleavage of the precursor (FIG. 1b) (Branda and Isaya, Prediction and identification of new natural substrates of the yeast mitochondrial intermediate peptidase. J. Biol. Chem., 270:27366-373, 1995), the inventors presume that P1A6F was cleaved precisely. (The exact cleavage point for C8A6F is unknown). As expected, the importation was dependent upon membrane potential, as treatment of isolated mitochondria with the uncoupler FCCP abolished processing of the P1A6F peptide (data not shown).

Similarly, use of anti-FLAG antibodies in Western-blot analyses of 293T HEK cells transiently transfected with pEF-BOS-P1A6F showed that P1A6F was imported and correctly processed into mitochondria in vivo (FIG. 3b). In the steady state, only about 18.5% of the precursor was imported and processed correctly. In mitochondria isolated from these cells, the majority of the precursor was sensitive to proteinase K treatment (FIG. 3b), implying that the precursors were either loosely attached to the mitochondrial outer membrane, or were attached but were not imported efficiently.

To show that mature A6F was assembled into the mitochondrial ATPase complex, the inventors carried out a native Western blot of solubilized mitochondrial complexes from 293T cells transfected with pBOS-IRES-P1A6F or with P1A6F inserted into AAV vectors. Detection with antibodies to FLAG and to subunit α of F₁-ATPase demonstrated co-migration of the immunoreactive bands in a complex of approximately 600 kD (FIG. 3c). This size corresponds to that of complex V, suggesting that rA6F was assembled into complex V.

Allotopic Expression of Recoded MTATP6 in mtDNA Mutant Cells

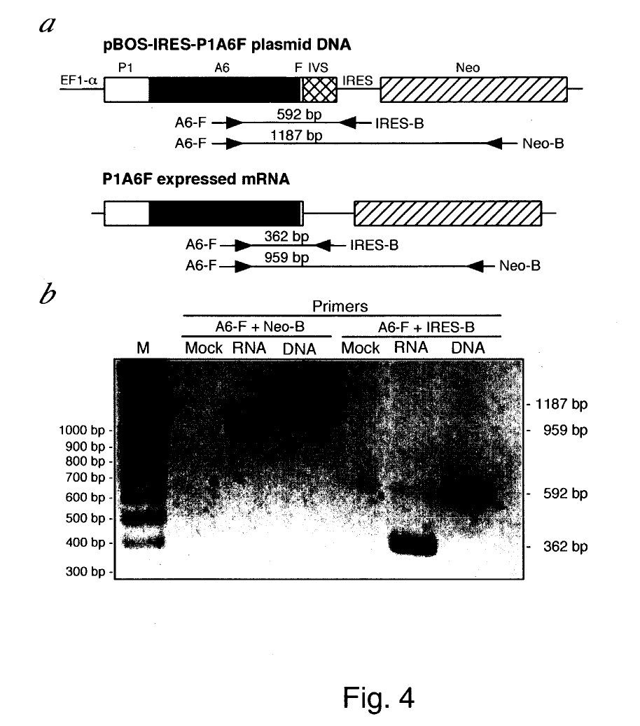

Homoplasmic cybrid lines harboring mutated mtDNA (100% 8993G) derived from an individual with MILS (Manfredi et al., Oligomycin induces a decrease in the cellular content of a pathogenic mutation in the human mitochondrial ATPase 6 gene. J. Biol. Chem., 274:9386-91, 1999) were transfected with pEF-BOS-IRES-P1A6F or mock-transfected with empty plasmid pCDNA3 (to introduce neo^{r}, the neomycin-resistance gene). After transfection, cells were grown in glucose-rich media containing the neomycin analog G418, to select for stably-transformed cells. The inventors have shown previously that cells with high levels of the 8993T→G mutation have a severe growth defect, as compared with wild-type cells, in medium containing galactose (rather than glucose) as the main carbon source for glycolysis and low levels of oligomycin (Manfredi et al., Oligomycin induces a decrease in the cellular content of a pathogenic mutation in the human mitochondrial ATPase 6 gene. J. Biol. Chem., 274:9386-91, 1999), a complex V inhibitor that binds specifically to the ATPase 6 polypeptide (Breen and et al., Mitochondrial DNA of two independent oligomycin-resistant Chinese hamster ovary cell lines contains a single nucleotide change in the ATPase 6 gene. J. Biol. Chem., 261:11680-85, 1986; John and Nagley, Amino acid substitutions in mitochondrial ATPase subunit 6 of Saccharomyces cerevisiae leading to oligomycin resistance. FEBS Lett., 207:79-83, 1986). Using this property as a basis for selection, G418-resistant cells were grown in medium containing galactose plus 0.1 ng ml⁻¹ oligomycin, after which the cells were allowed to recover in glucose-rich medium. Both the cybrids transfected with pBOS-IRES-P1A6F and those mock-transfected with pCDNA3 were subjected to the same selective growth conditions. In mutated cybrids, the selection presumably enriched for cells that expressed higher levels of P1A6F, and therefore had improved mitochondrial ATP synthesis. The inventors confirmed by RT-PCR that the cybrids expressed processed P1A6F mRNA (FIG. 4), and that P1A6F protein was localized to mitochondria soon after AAV infection (FIG. 2c). PCR-RFLP analysis confirmed that 100% of the mtDNA of the transfected cybrids contained the 8993T→G mutation (data not shown).

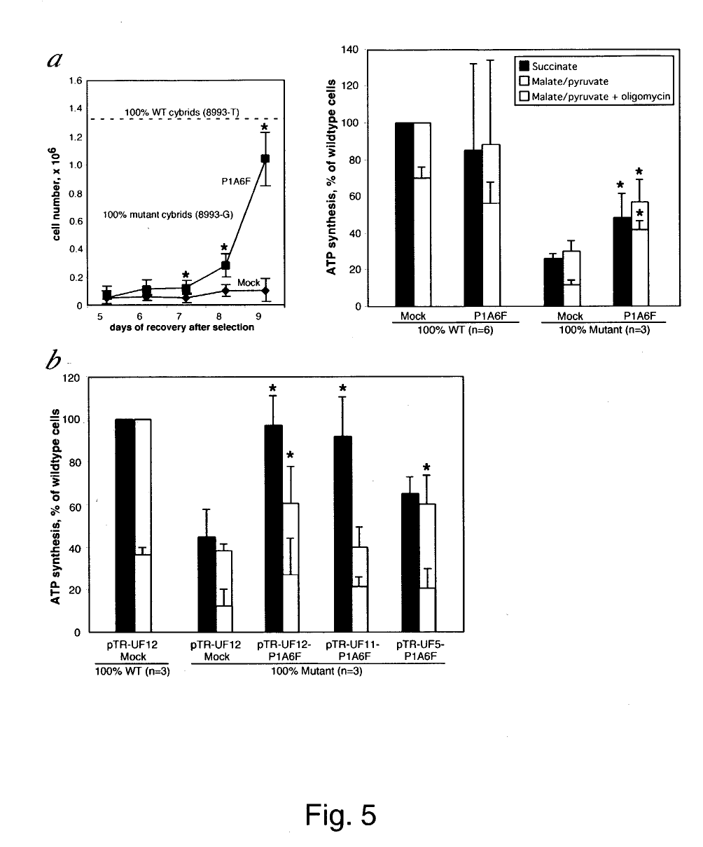

After selection in galactose/oligomycin, the mutated cybrids that were homoplasmic with respect to the 8993G mutation and that expressed P1A6F had a markedly-improved rate of growth, as compared with that of mock-transfected cybrids; the rate was significantly better within three days of the beginning of recovery in rich medium (FIG. 5a, left panel). In addition, there was also a significant increase in ATP synthesis in these cybrids, as compared with that in mock-transfected mutated cybrids selected in the same manner (FIG. 5a, right panel). In the cells stably transfected with pBOS-IRES-P1A6F, the increases in ATP synthesis were statistically significant (P<0.05, n=3) when both succinate and malate/pyruvate were used as respiratory substrates, and also when oligomycin (10 ng ml⁻¹) was added to the reaction to test for sensitivity to the inhibitor. These results imply that, in spite of the presence of the endogenous mtDNA-encoded mutated A6 polypeptides, at least some of the imported rA6F polypeptides had assembled successfully into functional complex V holoproteins.

The inventors obtained similar results in transient infections using the AAV constructs, albeit with a greater degree of variability. In mutated cybrids that were homoplasmic with respect to the 8993G mutation and transiently infected with three different parent rAAV plasmids expressing P1A6F, ATP synthesis was improved over that in mock-infected cells (FIG. 5b). The improvement in ATP synthesis was not statistically significant in all cases, however, presumably because of the inherent variability in the expression levels of P1A6F in transient-infection experiments. In particular, succinate-dependent ATP synthesis was significantly improved when P1A6F was inserted into the pTR-UF11 and pTR-UF12 AAV vectors; ATP synthesis also increased with the pTR-UF5 vector, but not significantly. Conversely, malate/pyruvate-dependent ATP synthesis improved significantly with the pTR-UF5 and pTR-UF12 vectors, but not with the pTR-UF11 vector (FIG. 5b).

The inventors have shown that allotopic expression of recoded MTATP6 can rescue a deficiency in ATP synthesis in transmitochondrial cybrids containing homoplasmic mtDNA with the 8993T→G mutation. Although C8A6F and P1A6F were imported into mitochondria, the efficiencies were relatively low. It may be that the MTS of C8 (25 amino acids) and P1 (61 amino acids) had difficulty in directing rapid and efficient importation of ATPase 6, a highly hydrophobic polypeptide, through the import machinery (Strub et al., The mitochondrial protein import motor. Biol. Chem., 381:943-49, 2000). The inventors note that, in a yeast system, the inability of the MTS of Neurospora crassa ATPase 9 (the homolog of human ATPc) to direct efficient import of a polypeptide specified by a recoded mtDNA-encoded yeast ATPase 8 gene was overcome by using a tandem duplication of the ATPase 9 MTS (Galanis et al., Duplication of leader sequence for protein targeting to mitochondria leads to increased import efficiency. FEBS Lett., 282:425-30, 1991).

Additionally, although mutational analyses of the analogous subunit a of Escherichia coli have shown that a FLAG epitope appended to the C terminus adversely affected ATP synthesis, the presence at the C terminus of either a slightly-altered version of FLAG or a His-6 epitope tag had no negative effects (Altendorf et al., Structure and function of the F(0) complex of the ATP synthase from Escherichia coli. J. Exp. Biol., 203:19-28, 2000; Jäger et al., Topology of subunit a of the Escherichia coli ATP synthase. Eur. J. Biochem., 251:122-32, 1998). It is possible that the slight (non-statistically significant) decrease in ATP synthesis that the inventors observed when wild-type cybrids were transfected with P1A6F (FIG. 5a) was a result of modifications engineered into the inventors' constructs. However, the potential impact of these modifications did not prevent ATP synthesis from increasing in mutant cybrids.

The improvement in ATP synthesis was more consistent and reproducible in cybrids stably transfected with pBOS-IRES-P1A6F and selected in galactose/oligomycin, than in those transiently infected with AAV. In particular, it is not clear why the improvements in ATP synthesis using malate/pyruvate as a substrate (an increase of approximately 50%, except for one vector which showed no improvement) were not as dramatic as the improvements that were observed when succinate was used (approximately double the value found in control cells). The inventors note, however, that electron flow through the respiratory chain is coupled to proton translocation from the matrix to the intermembrane space.

In cells harboring the 8993T→G mutation there is, perhaps unexpectedly, an increased membrane potential (Garcia et al., Structure, functioning, and assembly of the ATP synthase in cells from patients with the T8993G mitochondrial DNA mutation. Comparison with the enzyme in Rho⁰ cells completely lacking mtDNA. J. Biol. Chem., 275:11075-81, 2000), because proton flow through the F₀ portion of the ATPase is hindered (Schon et al., Pathogenesis of primary defects in mitochondrial ATP synthesis. Semin. Cell Dev. Biol., 12:441-48, 2001). This means that the respiratory chain must pump protons against a higher-than-normal gradient. Because the oxidation of one NADH molecule from complex I substrates (such as malate/pyruvate) forces the translocation of two more protons across the inner membrane, in comparison with the oxidation of one FADH₂ molecule from complex II substrates (such as succinate), it may be that it is easier for partially-rescued cells with the 8993T→G mutation to use succinate, rather than malate/pyruvate, to generate ATP. This phenomenon may be particularly crucial in transiently-infected cells that have not had sufficient time to adapt to a new metabolic state, e.g., by feedback regulation of uncoupling proteins. The inventors' results show, however, that the AAV gene-delivery system can be used to express ATPase 6 allotopically in mammalian cells, and that such constructs can potentially be used to express P1A6F in animal tissues. Accordingly, the inventors believe that allotopic expression of mtDNA-encoded polypeptides in mammalian cells may be used as a therapeutic approach for mitochondrial disorders for which there is currently no treatment.

### Example 7

Construction of Recorded ND4F and Adeno-Associated Virus Vectors

To construct the fusion gene containing the mitochondrial targeting sequences (MTSs) and epitope tag, the inventors created synthetic 80-mer oligonucleotide pairs in the nuclear genetic code, and codons prevalent in highly-expressed nuclear genes to conserve amino acid sequence. The synthetic oligonucleotides were overlapped by approximately 20 complementary nucleotides; these served as primers for polymerase chain reaction, with the high fidelity of Pfu Turbo DNA polymerase (Stratagene, La Jolla, Calif.), until the entire 1,377-nucleotide nuclear-encoded ND4 gene was constructed.

Using this technique, the inventors then fused the ND4 gene in-frame to the ATP1 or aldehyde dehydrogenase (Aldh) targeting sequences and to FLAG or green fluorescent protein (GFP) (Owen et al., Recombinant adenoassociated virus vector-based gene transfer for defects in oxidative metabolism. Hum. Gene Ther., 11:2067-78, 2000) epitope tags. Flanking XbaI (P1ND4Flag) or AflII and HindIII (AldhND4GFP) restriction sites were added, for cloning into AAV vectors. Base deletions and substitutions in the reading frame were corrected using the QuickChange in vitro mutagenesis kit (Stratagene, La Jolla, Calif.). The entire reading frame of the P1ND4Flag fusion gene was cloned in the XbaI sites of AAV plasmid vectors pTR-UF11 (regulated by the 381-bp cytomegalovirus immediate early gene enhancer 1,352-bp chicken β-actin promoter, exon 1 and intron 1). The AldhND4GFP was similarly constructed, but with flanking AflII and HindIII sites for cloning into pTRUF5 (Owen et al., Recombinant adenoassociated virus vector-based gene transfer for defects in oxidative metabolism. Hum. Gene Ther., 11:2067-78, 2000). COX8GFP was constructed and inserted into pTRUF5 (Owen et al., Recombinant adenoassociated virus vector-based gene transfer for defects in oxidative metabolism. Hum. Gene Ther., 11:2067-78, 2000).

To generate mitochondrially-targeted expression of P1ND4Flag and cytoplasmic-targeted expression of GFP in the same cell, the inventors used the pTR-UF12 vector that had P1ND4Flag linked to GFP via a 637-bp poliovirus internal ribosomal entry site (IRES). Both vectors have a splice donor/acceptor site from SV40 (16S/19S site), located just upstream of the coding sequence, to aid in the nuclear expression of and transport of the message. Visualization of cytoplasmic GFP enabled the inventors to identify conveniently those cells that were also expressing P1ND4Flag, which had been inserted upstream of the IRES.

The plasmids were amplified and purified by cesium chloride gradient centrifugation, and then packaged into recombinant AAV (rAAV) by transfection into human 293 cells using standard procedures. The rAAVs were titered by an infectious center assay (Hauswirth et al., Production and purification of recombinant adeno-associated virus. Methods Enzymol., 316:743-61, 2000).

### Example 8

Cell Culture and Viral Transfection

The study of the pathophysiology of mtDNA mutations has taken advantage of the use of transmitochondrial hybrid cell lines known as cybrids (King and Attardi. Mitochondria-mediated transformation of human rho⁰ cells. In: Attardi and Chomyn eds., Mitochondrial Biogenesis and Genetics, vol. 264 (San Diego, Calif.: Academic Press, 1996) 313-34). Cybrids are created by fusion of enucleated cells from patients with mutated mtDNA—in this case, the G11778A mutation—with cells that have permanently lost their mtDNA after chronic exposure to ethidium bromide. This procedure results in the production of a cell line with the mutated mtDNA of the patient, and the “neutral” nuclear DNA of the host cell line.

Homoplasmic osteosarcoma (143B.TK-)—derived cybrids containing wild-type (11778G) or mutated (11778A) mtDNA were constructed and cultured as previously reported (Vergani et al., MtDNA mutations associated with Leber's hereditary optic neuropathy: studies on cytoplasmic hybrid (cybrid) cells. Biochem. Biophys. Res. Commun., 210:880-88, 1995). For AAV infections, cybrids at approximately 80% confluency were transfected with 1 μg of DNA with TransIT Transfection Reagent (Mirus, Madison, Wis.) or 3.0×10⁷ AAV or rAAV viral particles in complete high-glucose medium. Selection in galactose was performed in 10 separate wells, and the cells were treated with selective medium for 3 days. Cells were trypsinized and counted using an automated Coulter (Hialeah, Fla.) Z-100 particle counter.

### Example 9

Immunological Techniques

For immunohistochemistry, the transfected cybrids were trypsinized and grown on glass slides. After the cells reached confluence, they were incubated for 30 min with 250 nM of the mitochondrial-specific fluorescent dye, MitoTracker Red (Molecular Probes, Eugene, Oreg.). Immunostaining with mouse monoclonal anti-FLAG M2 antibodies (Sigma, St. Louis, Mo.) or anti-GFP antibodies (ClonTech, Palo Alto, Calif.) was performed. Secondary anti-mouse Cy5 or Cy2, and anti-rabbit Cy2 (Jackson Immunochemicals, Bar Harbor, Me.), were used for immunodetection. Immunofluorescence was visualized in a Bio-Rad (Richmond, Calif.) confocal microscope. The collected digital images were pseudocolored red for MitoTracker, blue or green for FLAG, or green for GFP, then merged in red-green-blue (RGB) format for evaluation of co-localization.

For Western-blot analysis, sonicated proteins from total cellular lysates obtained from the transfected and restrictive-media-selected cells were electrophoresed through a 10% polyacrylamide gel, and electrotransferred to a polyvinylidene fluoride membrane (Bio-Rad). The membrane was immunostained with mouse monoclonal anti-FLAG M2 antibodies, and then with rabbit anti-mouse IgG alkaline-phosphatase-conjugated secondary antibodies. Immune complexes were detected by nitro-blue-tetrazolium chloride/5-bromo-4-chloro-3-indolylphosphate toludine salt (NBT/BCIP).

### Example 10

Oxidative Phosphorylation Assays

Assays of complex I (+III) activity were performed on P1ND4Flag and mock-transfected cybrids, in whole permeabilized cells, by the reduction of cytochrome c with nicotinamide adenine dinucleotide, and, additionally, in the presence of the inhibitor rotenone (Trounce et al., Assessment of mitochondrial oxidative phosphorylation in patient muscle biopsies, lymphoblasts, and transmitochondrial cell lines. Methods Enzymol., 264:484-509, 1996). ATP synthesis was measured by a luciferin-luciferase assay, in whole permeabilized cells, using the complex I substrates, malate and pyruvate, or the complex II substrate, succinate (Manfredi et al., Assay of mitochondrial ATP synthesis in animal cells. Methods Cell. Biol., 65:133-45. 2001). ATP synthesis with malate and pyruvate, or with succinate, was also measured after the addition of 10 ng/ml oligomycin to test for sensitivity to low doses of a specific ATPase inhibitor.

Discussed below are results obtained by the inventors in connection with the experiments of Examples 7-10:

Strategy for Allotopic Expression of ND4

To accomplish allotopic complementation, the inventors synthesized the full-length version of nuclear-encoded ND4, converting the “non-standard” codons, read by the mitochondrial genetic system, to the universal genetic code. The nucleotide sequence of the recoded ND4 was 73% homologous with the mitochondrial version of the ND4 gene, whereas the amino acid sequences encoded by both genes were identical. Therefore, the inventors' synthetic ND4 gene encodes for a “normal” ND4 protein that is identical to the ND4 protein synthesized within mitochondria; however, the inventors' recoded ND4 protein is synthesized in the cytoplasm.

To direct the import of the recoded ND4 protein into the mitochondria from the cytoplasm, the inventors added an MTS specifying the N-terminal region of either: (1) the P1 isoform of subunit c of human ATP synthase (ATPc), containing the entire 61-amino-acid MTS plus the first 5 amino acids of the mature P1 polypeptide (Higuti et al., Molecular cloning and sequence of two cDNAs for human subunit c of H(+)-ATP synthase in mitochondria. Biochim. Biophys. Acta., 1173:87-90, 1993); or (2) the Aldh containing the first 19 amino acids of the MTS (Ni et al., In vivo mitochondrial import. A comparison of leader sequence charge and structural relationships with the in vitro model resulting in evidence for cotranslational import. J. Biol. Chem., 274:12685-691, 1999). For detection of import, the inventors added to the C terminus of the P1ND4 gene the short FLAG epitope tag (24 nucleotides), or added to the AldhND4 gene the larger GFP tag (718 nucleotides).

Although the inventors began their mitochondrial import studies with GFP as the epitope tag, they ultimately switched to the much smaller FLAG tag. Even though GFP was successfully imported into mitochondria by an MTS fused to the N terminus, thereby making successful transfection easily detectable in living cell culture, import of the fusion protein was unsuccessful when GFP was fused to the C terminus of a recoded mitochondrial gene (ATP6 or ND6) (Owen et al., Recombinant adenoassociated virus vector-based gene transfer for defects in oxidative metabolism. Hum. Gene Ther., 11:2067-78, 2000).

To achieve stable and efficient expression of the fusion gene in cells, the inventors inserted P1ND4Flag into AAV vectors, pTR-UF11 and pTR-UF12. Transgene expression in both vectors is driven by the chicken β-actin promoter and cytomegalovirus enhancer. In addition, pTR-UF12 also contains an IRES linked to GFP, for identification of transfected cells in living cell cultures. Thus, GFP (lacking a MTS) is expressed only in the cytoplasm, whereas the P1ND4Flag fusion protein is expressed in the mitochondria of the same cell. Unlike plasmid transfection that requires the addition of chemical reagents to facilitate DNA entry into cells, and produces only transient and somewhat inefficient expression of the introduced gene, viral-mediated gene transfer permits efficient delivery of genes into cells for assays of transgene function (Bai et al., Lack of complex I activity in human cells carrying a mutation in mtDNA-encoded ND4 subunit is corrected by the Saccharomyces cerevisiae NADHquinone oxidoreductase (NDI1) gene. J. Biol. Chem., 276:38808-813, 2001). Moreover, in the case of AAV, the transferred DNA sequences may be integrated stably into the chromosomal DNA of the target cell, for long-term expression of the transgene in vivo in living cells, organs, and tissues (Guy et al., Reporter expression persists 1 year after adeno-associated virus-mediated gene transfer to the optic nerve. Arch. Ophthalmol., 117:929-37, 1999; Guy et al., Adeno-associated viral-mediated catalase expression suppresses optic neuritis in experimental allergic encephalomyelitis. Proc. Natl Acad. Sci. USA, 95:13847-852, 1998).

Detection of Allotopic Expression in Cells Containing Mutated Mitochondrial DNA

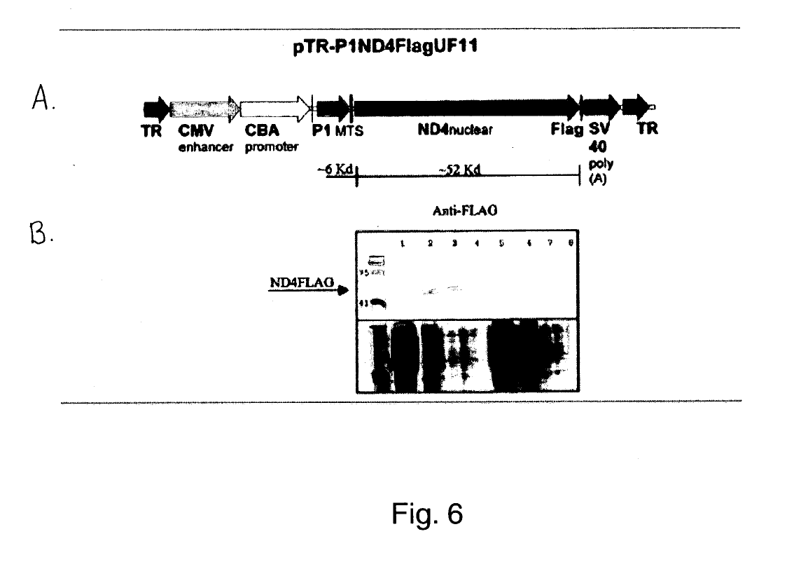

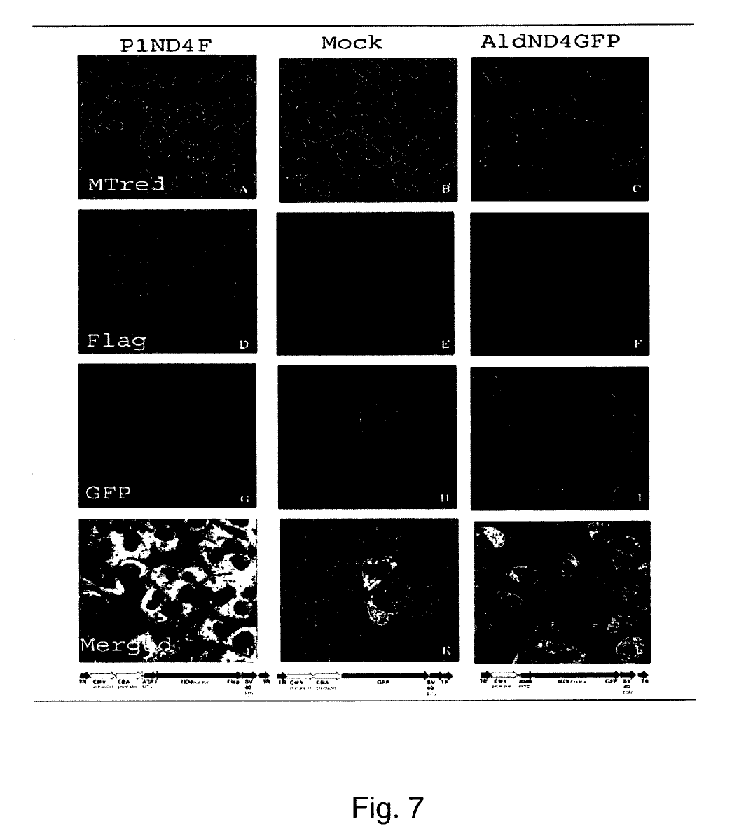

Homoplasmic human cybrid cells, containing the mitochondria of patients harboring the G11778A mutation in mtDNA, and transfected with rAAV containing the P1ND4Flag fusion gene, expressed the fusion polypeptide (FIG. 6). The ATPc MTS directed the allotopically-expressed ND4F polypeptide into mitochondria. Immunocytochemistry to detect the FLAG epitope, inserted at the C terminus of the imported ND4, showed a typical punctate mitochondrial pattern that co-localized with the mitochondrion-specific dye, MitoTracker Red, thereby implying that the recoded ND4Flag was imported into mitochondria (FIG. 7). Cells transfected with P1ND4Flag in AAV vector, UF-11, showed mitochondrially-targeted FLAG (FIG. 7D) co-localized with MitoTracker Red (FIG. 7A), as demonstrated in the merged panel (FIG. 7J) of FIG. 7. Cells transfected with P1ND4Flag in AAV vector, UF-12, that contained the IRES linked to GFP, showed mitochondrially-targeted FLAG and cytoplasmic GFP in the same cell. Cells mock-transfected with AAV vector, UF-11, driving GFP expression in the place of the P1ND4Flag gene, exhibited diffuse cytoplasmic staining of GFP only (FIG. 7H). Lastly, when ND4 with the Aldh MTS was linked to GFP, rather than to FLAG, the ND4GFP fusion did have a punctate staining pattern, mimicking import into mitochondria (FIG. 7I). However, relatively poor co-localization of GFP with MitoTracker Red (FIG. 7I) suggested that this fusion protein was not imported.

Allotopic ND4 Improves Cybrid Cell Survival

Although P1ND4Flag was expressed and imported into mitochondria, the inventors queried whether allotopic complementation with this protein would improve the defective oxidative phosphorylation of LHON. To answer this question, homoplasmic cybrid cells harboring mutant mtDNA (i.e., 100% G11778A derived from a patient with LHON inserted into a neutral nuclear background) were transfected with rAAV containing the P1ND4Flag, or mock-transfected with the same AAV plasmid lacking the allotropic insert and expressing GFP (UF-11). Immediately after the transfection, cells were grown in glucose-rich media for 3 days, and then placed in a glucose-free medium containing galactose as the main carbon source for glycolysis. This medium forces the cells to rely predominantly on oxidative phosphorylation to produce ATP (Reitzer et al., Evidence that glutamine, not sugar, is the major energy source for cultured HeLa cells. J. Biol. Chem., 254:2669-76, 1979).

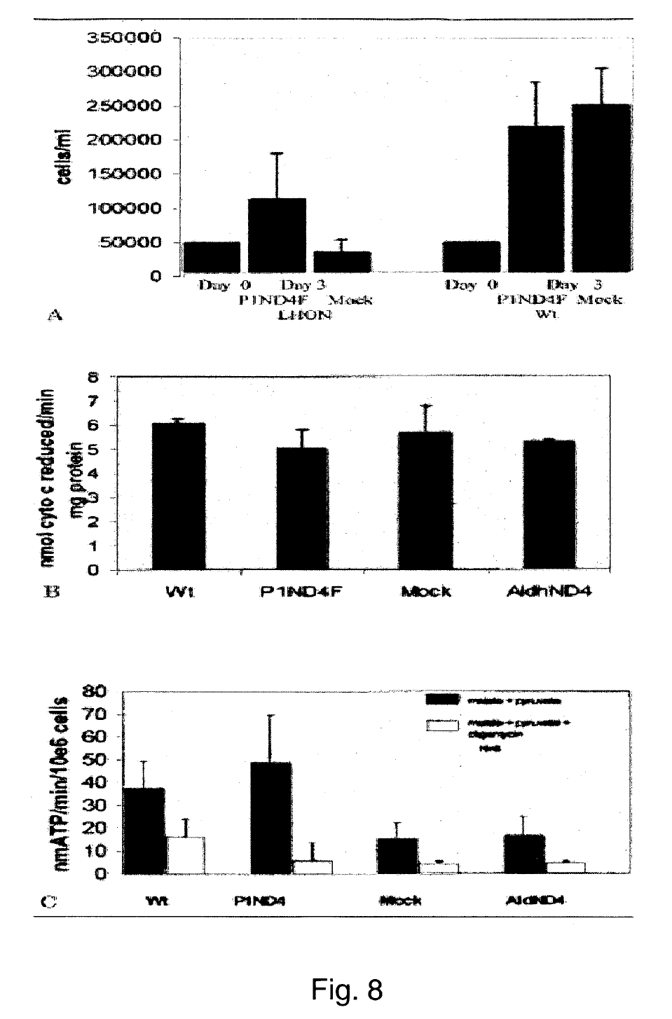

Cells harboring complex I mutations have a severe growth defect, when compared with wild-type cells in such medium (Bai et al., Lack of complex I activity in human cells carrying a mutation in mtDNA-encoded ND4 subunit is corrected by the Saccharomyces cerevisiae NADHquinone oxidoreductase (NDI1) gene. J. Biol. Chem., 276:38808-813, 2001). The inventors found that cybrid-cell survival after 3 days in the glucose-deficient galactose medium was three-fold greater for the allotopically-transfected P1ND4Flag cybrids than for the cybrids transfected with the mock AAV (p<0.001; FIG. 8A). Apparently, in the mutated cybrids, this selection enriched for cells that expressed higher levels of P1ND4Flag, suggesting that these cells likely had improved oxidative phosphorylation.

Oxidative Phosphorylation Deficiency Rescued by Allotopic ND4

Consistent with the finding that spectrophotometric assays of complex I activity do not discriminate between wild-type cells and G11778A mutant cybrids (Majander et al., Electron transfer properties of NADH:ubiquinone reductase in the ND1/3460 and the ND4/11778 mutations of the Leber hereditary optic neuroretinopathy (LHON). FEBS Lett., 292:289-92, 1991; Larsson et al., Leber's hereditary optic neuropathy and complex I deficiency in muscle. Ann. Neurol., 30:701-08, 1991; Brown et al., Functional analysis of lymphoblast and cybrid mitochondria containing the 3460, 11778, or 14484 Leber's hereditary optic neuropathy mitochondrial DNA mutation. J. Biol. Chem., 275:39831-836, 2000; Andreu et al., Exercise intolerance due to a nonsense mutation in the mtDNA ND4 gene. Ann. Neurol., 45:820-23, 1999; Hofhaus et al., Respiration and growth defects in transmitochondrial cell lines carrying the 11778 mutation associated with Leber's hereditary optic neuropathy. J. Biol. Chem., 271:13155-161, 1996), transfection with P1ND4Flag did not increase complex I activity (FIG. 8B). These results are in accord with published observations that the impact of the G11778A LHON mutation on complex-I-specific activity in cell lines appears to be mild (Brown et al., Functional analysis of lymphoblast and cybrid mitochondria containing the 3460, 11778, or 14484 Leber's hereditary optic neuropathy mitochondrial DNA mutation. J. Biol. Chem., 275:39831-836, 2000; Hofhaus et al., Respiration and growth defects in transmitochondrial cell lines carrying the 11778 mutation associated with Leber's hereditary optic neuropathy. J. Biol. Chem., 271:13155-161, 1996). Therefore, the inventors focused on changes in ATP synthesis using malate and pyruvate as complex I substrates for oxidative phosphorylation (FIG. 8C) (Larsson et al., Leber's hereditary optic neuropathy and complex I deficiency in muscle. Ann. Neurol., 30:701-08, 1991).

It has been shown that respiration of G11778A cell lines is reduced with complex I substrates, but may be increased with complex II substrates—due, perhaps, to compensatory regulation of the nuclear-encoded complex II (Majander et al., Electron transfer properties of NADH:ubiquinone reductase in the ND1/3460 and the ND4/11778 mutations of the Leber hereditary optic neuroretinopathy (LHON). FEBS Lett., 292:289-92, 1991; Larsson et al., Leber's hereditary optic neuropathy and complex I deficiency in muscle. Ann. Neurol., 30:701-08, 1991; Yen et al., Compensatory elevation of complex II activity in Leber's hereditary optic neuropathy. Br. J. Ophthalmol., 80:78-81, 1996). Consistent with these observations, the inventors found that, relative to the wild-type cell line with normal mtDNA, cybrid cells containing the LHON G11778A mutation in mtDNA showed a 60% reduction in the rate of complex-I-dependent ATP synthesis (p<0.005) (Yen et al., Energy charge is not decreased in lymphocytes of patients with Leber's hereditary optic neuropathy with the 11,778 mutation. J. Neuroophthalmol., 18:84-85, 1998; Majander et al., Mutations in subunit 6 of the F1F0-ATP synthase cause two entirely different diseases. FEBS Lett., 412:351-54, 1997; Lodi et al., In vivo skeletal muscle mitochondrial function in Leber's hereditary optic neuropathy assessed by 31P magnetic resonance spectroscopy. Ann. Neurol., 42:573-79, 1997). Moreover, using the complex II substrate, succinate, that bypasses the mutated complex I, the inventors found that ATP synthesis in G11778A cybrids increased five-fold (82 nm ATP/min/10⁶ cells with succinate vs. 15 nm ATP/min/10⁶ cells with malate and pyruvate; p<0.02). However, in the wild-type cell line containing normal mtDNA, the rates of ATP synthesis obtained with either complex I or complex II substrates were virtually identical (30.8 nm ATP/min/10⁶ cells with succinate vs. 31.4 nm ATP/min/10⁶ cells with malate and pyruvate).

Although complex-II-dependent ATP synthesis was actually increased more than two-fold (p<0.05) in the inventors' LHON cybrids, relative to the wild-type cell line, this finding was likely compensatory, as previously demonstrated (Majander et al., Electron transfer properties of NADH:ubiquinone reductase in the ND1/3460 and the ND4/11778 mutations of the Leber hereditary optic neuroretinopathy (LHON). FEBS Lett., 292:289-92, 1991; Larsson et al., Leber's hereditary optic neuropathy and complex I deficiency in muscle. Ann. Neurol., 30:701-08, 1991; Yen et al., Compensatory elevation of complex II activity in Leber's hereditary optic neuropathy. Br. J. Ophthalmol., 80:78-81, 1996). Therefore, the inventors focused their attention on the main problem, the deficiency in complex-I-dependent ATP synthesis induced by the G11778A mutation in the mitochondrial gene for complex I. Such substantial reductions in ATP synthesis likely contribute to the development of optic neuropathy in LHON patients with the G11778A mutation. However, the inventors wondered whether allotopic expression of a normal ND4 gene would rescue the substantial deficiency in complex-I-dependent ATP synthesis of LHON cybrids.

Indeed, relative to G11778A cybrids transfected with a mock AAV vector lacking the P1ND4Flag gene, P1ND4Flag-complemented G11778A cybrids showed a three-fold increase in the rate of complex-I-dependent ATP synthesis. This degree of recovery led to levels of ATP synthesis that were virtually indistinguishable from those of the corresponding wild-type cell line containing normal mtDNA. Although the level of transfection by AAV containing P1ND4Flag is somewhat variable, as shown by higher standard deviations obtained with this construct, the differences between P1ND4Flag and mock-transfected cybrids were statistically significant (p<0.02); thus, P1ND4Flag has a major impact on ATP synthesis. In contrast, when the AldhND4GFP construct was tested, cytoplasmic expression of ND4 had no impact on ATP levels, as predicted by the lack of mitochondrial import (FIG. 7I).

While the foregoing invention has been described in some detail for purposes of clarity and understanding, it will be appreciated by one skilled in the art, from a reading of the disclosure, that various changes in form and detail can be made without departing from the true scope of the invention in the appended claims.

What is claimed is:

1. A method for introducing a functional peptide encoded by a non-nuclear nucleic acid sequence into an organelle, comprising the steps of:
(a) preparing a nucleic-acid construct comprising a non-nuclear nucleic acid sequence encoding the peptide and a nucleic acid sequence encoding an organelle-targeting signal;
(b) introducing the nucleic-acid construct into a eukaryotic cell to produce a transformed cell, wherein the eukaryotic cell is derived from algae, an animal, a multicellular or other non-yeast fungus, or protozoa; and
(c) expressing the nucleic-acid construct from the nucleus of the transformed cell.

2. The method of claim 1, further comprising the step of mutagenizing the non-nuclear nucleic acid sequence encoding the peptide, if necessary, before step (a), to render the non-nuclear nucleic acid sequence compatible with the universal genetic code.

3. The method of claim 2, wherein the organelle is a mitochondrion.

4. The method of claim 2, wherein the peptide is a mitochondrial-DNA-encoded (mtDNA-encoded) peptide.

5. The method of claim 4, wherein the mtDNA-encoded peptide is ATPase 6 subunit of F₀F₁-ATP synthase or ND4 subunit of complex I.

6. The method of claim 2, wherein the organelle-targeting signal is selected from the group consisting of the N-terminal region of human cytochrome c oxidase subunit VIII, the N-terminal region of the P1 isoform of subunit c of human ATP synthase, and the N-terminal region of the aldehyde dehydrogenase targeting sequence.

7. The method of claim 2, wherein the nucleic-acid construct is introduced into the eukaryotic cell by a method selected from the group consisting of electroporation, DEAE Dextran transfection, calcium phosphate transfection, cationic liposome fusion, protoplast fusion, creation of an in vivo electrical field, DNA-coated microprojectile bombardment, injection with a recombinant replication-defective virus, homologous recombination, ex vivo gene therapy, a viral vector, and naked DNA transfer.

8. The method of claim 2, wherein the eukaryotic cell is a mammalian cell.

9. The method of claim 8, wherein the cell is a human cell.

10. The method of claim 9, where the cell is a human 293T HEK cell.

11. The method of claim 2, wherein the nucleic-acid construct further comprises a nucleic acid sequence encoding a detectable marker.

12. The method of claim 11, wherein the detectable marker is a FLAG epitope or green fluorescent protein (GFP).

13. The method of claim 2, wherein the organelle is a mitochondrion; the peptide is a mitochondrial-DNA-encoded (mtDNA-encoded) peptide; the organelle-targeting signal is selected from the group consisting of the N-terminal region of human cytochrome c oxidase subunit VIII, the N-terminal region of the P1 isoform of subunit c of human ATP synthase, and the N-terminal region of the aldehyde dehydrogenase targeting sequence; and the eukaryotic cell is a mammalian cell.

14. The method of claim 13, wherein the mtDNA-encoded peptide is ATPase 6 subunit of F₀F₁-ATP synthase, and the organelle-targeting signal is the N-terminal region of human cytochrome c oxidase subunit VIII or the N-terminal region of the P1 isoform of subunit c of human ATP synthase.

15. The method of claim 13, wherein the mtDNA-encoded peptide is ND4 subunit of complex I, and the organelle-targeting signal is the N-terminal region of the P1 isoform of subunit c of human ATP synthase or the N-terminal region of the aldehyde dehydrogenase targeting sequence.

16. The method of claim 2, wherein the eukaryotic cell is in, or is introduced into, a mammal.

17. The method of claim 16, wherein the mammal is a human.

18. The method of claim 13, wherein the mammalian cell is in, or is introduced into, a human.

19. The method of claim 18, wherein the human has a mitochondrial disorder.

20. The method of claim 19, wherein the mitochondrial disorder is associated with a mutation in mtDNA.

21. The method of claim 20, wherein the mutation is a point mutation.

22. The method of claim 20, wherein the mitochondrial disorder is selected from the group consisting of FBSN (familial bilateral striatal necrosis), LHON (Leber hereditary optic neuropathy), MILS (maternally-inherited Leigh syndrome), and NARP (neuropathy, ataxia, and retinitis pigmentosa).

23. The method of claim 22, wherein the mtDNA-encoded peptide is wild-type ATPase 6 subunit of F₀F₁-ATP synthase or wild-type ND4 subunit of complex I.

24. A method for introducing a functional peptide encoded by a mitochondrial DNA (mtDNA) sequence into an organelle, comprising the steps of:
(a) preparing a nucleic-acid construct comprising an mtDNA sequence encoding the peptide and a nucleic acid sequence encoding an organelle-targeting signal;
(b) introducing the nucleic-acid construct into a eukaryotic cell to produce a transformed cell, wherein the eukaryotic cell is derived from algae, an animal, a plant, a multicellular or other non-yeast fungus, or protozoa; and
(c) expressing the nucleic-acid construct from the nucleus of the transformed cell.

25. The method of claim 24, further comprising the step of mutagenizing the mtDNA sequence encoding the peptide, before step (a), to render the mtDNA sequence compatible with the universal genetic code.

26. The method of claim 25, wherein the organelle is a mitochondrion.

27. The method of claim 25, wherein the mtDNA-encoded peptide is ATPase 6 subunit of F₀F₁-ATP synthase or ND4 subunit of complex I.

28. The method of claim 25, wherein the organelle-targeting signal is selected from the group consisting of the N-terminal region of human cytochrome c oxidase subunit VIII, the N-terminal region of the P1 isoform of subunit c of human ATP synthase, and the N-terminal region of the aldehyde dehydrogenase targeting sequence.

29. The method of claim 25, wherein the nucleic-acid construct is introduced into the eukaryotic cell by a method selected from the group consisting of electroporation, DEAE Dextran transfection, calcium phosphate transfection, cationic liposome fusion, protoplast fusion, creation of an in vivo electrical field, DNA-coated microprojectile bombardment, injection with a recombinant replication-defective virus, homologous recombination, ex vivo gene therapy, a viral vector, and naked DNA transfer.

30. The method of claim 25, wherein the eukaryotic cell is a mammalian cell.

31. The method of claim 30, wherein the cell is a human cell.

32. The method of claim 31, wherein the cell is a human 293T HEK cell.

33. The method of claim 25, wherein the nucleic-acid construct further comprises a nucleic acid sequence encoding a detectable marker.

34. The method of claim 33, wherein the detectable marker is a FLAG epitope or green fluorescent protein (GFP).

35. The method of claim 25, wherein the organelle is a mitochondrion; the organelle-targeting signal is selected from the group consisting of the N-terminal region of human cytochrome c oxidase subunit VIII, the N-terminal region of the P1 isoform of subunit c of human ATP synthase, and the N-terminal region of the aldehyde dehydrogenase targeting sequence; and the eukaryotic cell is a mammalian cell.

36. The method of claim 35, wherein the mtDNA-encoded peptide is ATPase 6 subunit of F₀F₁-ATP synthase, and the organelle-targeting signal is the N-terminal region of human cytochrome c oxidase subunit VIII or the N-terminal region of the P1 isoform of subunit c of human ATP synthase.

37. The method of claim 35, wherein the mtDNA-encoded peptide is ND4 subunit of complex I, and the organelle-targeting signal is the N-terminal region of the P1 isoform of subunit c of human ATP synthase or the N-terminal region of the aldehyde dehydrogenase targeting sequence.

38. The method of claim 25, wherein the eukaryotic cell is in, or is introduced into, a mammal.

39. The method of claim 38, wherein the mammal is a human.

40. The method of claim 35, wherein the mammalian cell is in, or is introduced into, a human.

41. The method of claim 40, wherein the human has a mitochondrial disorder.

42. The method of claim 41, wherein the mitochondrial disorder is associated with a mutation in mtDNA.

43. The method of claim 42, wherein the mutation is a point mutation.

44. The method of claim 42, wherein the mitochondrial disorder is selected from the group consisting of FBSN (familial bilateral striatal necrosis), LHON (Leber hereditary optic neuropathy), MILS (maternally-inherited Leigh syndrome), and NARP (neuropathy, ataxia, and retinitis pigmentosa).

45. The method of claim 44, wherein the mtDNA-encoded peptide is wild-type ATPase 6 subunit of F₀F₁-ATP synthase or wild-type ND4 subunit of complex I.

46. A method for correcting a phenotypic deficiency in a mammal that results from a mutation in a peptide-encoding sequence of the mammal's mitochondrial DNA (mtDNA), comprising the steps of:
(a) identifying the peptide-encoding sequence of the mammal's mtDNA in which the mutation occurs;
(b) preparing a nucleic-acid construct comprising the peptide-encoding sequence of mtDNA and a nucleic acid sequence encoding a mitochondrial-targeting signal, wherein the peptide-encoding sequence of mtDNA encodes a wild-type peptide;
(c) introducing the nucleic-acid construct into a mammalian cell to produce a transformed cell; and
(d) expressing the nucleic-acid construct from the nucleus of the transformed cell.

47. The method of claim 46, further comprising the step of mutagenizing the peptide-encoding sequence of mtDNA, before step (b), to render the mtDNA sequence compatible with the universal genetic code.

48. The method of claim 46, wherein the mtDNA-encoded peptide is ATPase 6 subunit of F₀F₁-ATP synthase or ND4 subunit of complex I.

49. The method of claim 46, wherein the mitochondrial-targeting signal is selected from the group consisting of the N-terminal region of human cytochrome c oxidase subunit VIII, the N-terminal region of the P1 isoform of subunit c of human ATP synthase, and the N-terminal region of the aldehyde dehydrogenase targeting sequence.

50. The method of claim 46, wherein the nucleic-acid construct is introduced into the mammalian cell by a method selected from the group consisting of electroporation, DEAE Dextran transfection, calcium phosphate transfection, cationic liposome fusion, protoplast fusion, creation of an in vivo electrical field, DNA-coated microprojectile bombardment, injection with a recombinant replication-defective virus, homologous recombination, ex vivo gene therapy, a viral vector, and naked DNA transfer.

51. The method of claim 46, wherein the mammalian cell is a human cell.

52. The method of claim 49, wherein the mtDNA-encoded peptide is ATPase 6 subunit of F₀F₁-ATP synthase, and the mitochondrial-targeting signal is the N-terminal region of human cytochrome c oxidase subunit VIII or the N-terminal region of the P1 isoform of subunit c of human ATP synthase.

53. The method of claim 49, wherein the mtDNA-encoded peptide is ND4 subunit of complex I, and the mitochondrial-targeting signal is the N-terminal region of the P1 isoform of subunit c of human ATP synthase or the N-terminal region of the aldehyde dehydrogenase targeting sequence.

54. The method of claim 46, wherein the mammalian cell is in, or is introduced into, a human.

55. The method of claim 54, wherein the human has a mitochondrial disorder.

56. The method of claim 55, wherein the mitochondrial disorder is associated with a mutation in mtDNA.

57. The method of claim 56, wherein the mutation is a point mutation.

58. The method of claim 56, wherein the mitochondrial disorder is selected from the group consisting of FBSN (familial bilateral striatal necrosis), LHON (Leber hereditary optic neuropathy), MILS (maternally-inherited Leigh syndrome), and NARP (neuropathy, ataxia, and retinitis pigmentosa).

59. The method of claim 58, wherein the mtDNA-encoded peptide is wild-type ATPase 6 subunit of F₀F₁-ATP synthase or wild-type ND4 subunit of complex I.

60. A method for treating a mitochondrial disorder in a subject in need of treatment therefor, comprising administering to the subject a mitochondrial-DNA-encoded (mtDNA-encoded) peptide in an amount effective to treat the mitochondrial disorder.

61. The method of claim 60, wherein the mtDNA-encoded peptide is administered to the subject by introducing into one or more cells of the subject a mitochondrial DNA (mtDNA) sequence encoding the peptide, in a manner permitting expression of the peptide.

62. The method of claim 60, wherein the mtDNA-encoded peptide is administered to the subject by a method comprising the steps of:
(a) obtaining an mtDNA sequence encoding the peptide;
(b) mutagenizing the mtDNA sequence to render it compatible with the universal genetic code, thereby producing mutagenized mtDNA;
(c) preparing a nucleic-acid construct comprising the mutagenized mtDNA and a nucleic acid sequence encoding a mitochondrial-targeting signal;
(d) introducing the nucleic-acid construct into one or more cells of the subject; and
(e) in at least one cell of the subject into which the nucleic-acid construct is introduced, expressing the nucleic-acid construct from the nucleus of the cell.

63. The method of claim 62, wherein step (d) is performed ex vivo.

64. The method of claim 60, wherein the mtDNA-encoded peptide is ATPase 6 subunit of F₀F₁-ATP synthase or ND4 subunit of complex I.

65. The method of claim 62, wherein the mitochondrial-targeting signal is selected from the group consisting of the N-terminal region of human cytochrome c oxidase subunit VIII, the N-terminal region of the P1 isoform of subunit c of human ATP synthase, and the N-terminal region of the aldehyde dehydrogenase targeting sequence.

66. The method of claim 62, wherein the nucleic-acid construct is introduced into one or more cells of the subject by a method selected from the group consisting of electroporation, DEAE Dextran transfection, calcium phosphate transfection, cationic liposome fusion, protoplast fusion, creation of an in vivo electrical field, DNA-coated microprojectile bombardment, injection with a recombinant replication-defective virus, homologous recombination, ex vivo gene therapy, a viral vector, and naked DNA transfer.

67. The method of claim 60, wherein the subject is a mammal.

68. The method of claim 67, wherein the mammal is a human.

69. The method of claim 62, wherein the mtDNA-encoded peptide is ATPase 6 subunit of F₀F₁-ATP synthase, and the mitochondrial-targeting signal is the N-terminal region of human cytochrome c oxidase subunit VIII or the N-terminal region of the P1 isoform of subunit c of human ATP synthase.

70. The method of claim 62, wherein the mtDNA-encoded peptide is ND4 subunit of complex I, and the mitochondrial-targeting signal is the N-terminal region of the P1 isoform of subunit c of human ATP synthase or the N-terminal region of the aldehyde dehydrogenase targeting sequence.

71. The method of claim 60, wherein the mitochondrial disorder is associated with a mutation in mtDNA.

72. The method of claim 71, wherein the mutation is a point mutation.

73. The method of claim 71, wherein the mitochondrial disorder is selected from the group consisting of FBSN (familial bilateral striatal necrosis), LHON (Leber hereditary optic neuropathy), MILS (maternally-inherited Leigh syndrome), and NARP (neuropathy, ataxia, and retinitis pigmentosa).

74. The method of claim 73, wherein the mtDNA-encoded peptide is wild-type ATPase 6 subunit of F₀F₁-ATP synthase or wild-type ND4 subunit of complex I.

75. An expression vector that is useful for introducing a functional peptide encoded by a mitochondrial DNA (mtDNA) sequence into a mitochondrion, comprising:
(a) a nucleic acid sequence encoding ATPase 6 subunit of F₀F₁-ATP synthase or ND4 subunit of complex I, wherein the nucleic acid sequence is compatible with the universal genetic code; and
(b) a nucleic acid sequence encoding a mitochondrial-targeting signal, wherein the mitochondrial-targeting signal is selected from the group consisting of the N-terminal region of human cytochrome c oxidase subunit VIII, the N-terminal region of the P1 isoform of subunit c of human ATP synthase, and the N-terminal region of the aldehyde dehydrogenase targeting sequence.

76. The expression vector of claim 75, further comprising a nucleic acid sequence encoding a detectable marker.

77. The expression vector of claim 76, wherein the detectable marker is a FLAG epitope or green fluorescent protein (GFP).

78. The expression vector of claim 75, wherein the mitochondrial-targeting signal is the N-terminal region of human cytochrome c oxidase subunit VIII or the N-terminal region of the P1 isoform of subunit c of human ATP synthase.

79. The expression vector of claim 75, wherein the mitochondrial-targeting signal is the N-terminal region of the P1 isoform of subunit c of human ATP synthase or the N-terminal region of the aldehyde dehydrogenase targeting sequence.

80. The expression vector of claim 75, wherein the vector is selected from the group consisting of a bicistronic vector, a plasmid vector, and an adeno-associated virus (AAV) vector.

81. A eukaryotic cell transformed by the expression vector of claim 75, wherein the eukaryotic cell is derived from algae, an animal, a plant, a multicellular or other non-yeast fungus, or protozoa.

82. A eukaryotic cell transformed by the expression vector of claim 77, wherein the eukaryotic cell is derived from algae, an animal, a plant, a multicellular or other non-yeast fungus, or protozoa.

83. A eukaryotic cell transformed by an expression vector that is useful for introducing a functional peptide encoded by a non-nuclear nucleic acid sequence into an organelle, wherein the eukaryotic cell is derived from algae, an animal, a multicellular or other non-yeast fungus, or protozoa, and the expression vector comprises:
(a) a non-nuclear nucleic acid sequence encoding the peptide, wherein the nucleic acid sequence is compatible with the universal genetic code; and
(b) a nucleic acid sequence encoding an organelle-targeting signal.

84. The eukaryotic cell of claim 83, wherein the cell expresses the peptide.

85. The eukaryotic cell of claim 83, which is a mammalian cell.

86. The eukaryotic cell of claim 85, which is a human cell.

87. The eukaryotic cell of claim 83, which is selected from the group consisting of a clonal cell, a stem cell, and a progenitor cell.

88. The eukaryotic cell of claim 83, wherein the peptide is a mitochondrial-DNA-encoded (mtDNA-encoded) peptide.

89. The eukaryotic cell of claim 88, wherein the mtDNA-encoded peptide is ATPase 6 subunit of F₀F₁-ATP synthase or ND4 subunit of complex I.

90. The eukaryotic cell of claim 83, wherein the organelle-targeting signal is selected from the group consisting of the N-terminal region of human cytochrome c oxidase subunit VIII, the N-terminal region of the P1 isoform of subunit c of human ATP synthase, and the N-terminal region of the aldehyde dehydrogenase targeting sequence.

91. The eukaryotic cell of claim 83, wherein the expression vector transforms the cell by a method selected from the group consisting of electroporation, DEAE Dextran transfection, calcium phosphate transfection, cationic liposome fusion, protoplast fusion, creation of an in vivo electrical field, DNA-coated microprojectile bombardment, injection with a recombinant replication-defective virus, homologous recombination, ex vivo gene therapy, a viral vector, and naked DNA transfer.

92. The eukaryotic cell of claim 83, wherein the expression vector further comprises a nucleic acid sequence encoding a detectable marker.

93. The eukaryotic cell of claim 92, wherein the detectable marker is a FLAG epitope or green fluorescent protein (GFP).

94. The eukaryotic cell of claim 85, wherein the peptide is a mitochondrial-DNA-encoded (mtDNA-encoded) peptide, and the organelle-targeting signal is selected from the group consisting of the N-terminal region of human cytochrome c oxidase subunit VIII, the N-terminal region of the P1 isoform of subunit c of human ATP synthase, and the N-terminal region of the aldehyde dehydrogenase targeting sequence.

95. The eukaryotic cell of claim 94, wherein the mtDNA-encoded peptide is ATPase 6 subunit of F₀F₁-ATP synthase, and the organelle-targeting signal is the N-terminal region of human cytochrome c oxidase subunit VIII or the N-terminal region of the P1 isoform of subunit c of human ATP synthase.

96. The eukaryotic cell of claim 94, wherein the mtDNA-encoded peptide is ND4 subunit of complex I, and the organelle-targeting signal is the N-terminal region of the P1 isoform of subunit c of human ATP synthase or the N-terminal region of the aldehyde dehydrogenase targeting sequence.

97. The eukaryotic cell of claim 83, wherein the expression vector is selected from the group consisting of a bicistronic vector, a plasmid vector, and an adeno-associated virus (AAV) vector.

98. A clonal cell strain comprising the transformed eukaryotic cell of claim 83.

99. A eukaryotic cell transformed by an expression vector that is useful for introducing a functional peptide encoded by a mitochondrial DNA (mtDNA) sequence into an organelle, wherein the eukaryotic cell is derived from algae, an animal, a multicellular or other non-yeast fungus, a plant, or protozoa, and the expression vector comprises:
(a) an mtDNA sequence encoding the peptide, wherein the mtDNA sequence is compatible with the universal genetic code; and
(b) a nucleic acid sequence encoding an organelle-targeting signal.

100. The eukaryotic cell of claim 99, wherein the cell expresses the peptide.

101. The eukaryotic cell of claim 99, which is a mammalian cell.

102. The eukaryotic cell of claim 101, which is a human cell.

103. The eukaryotic cell of claim 99, which is selected from the group consisting of a clonal cell, a stem cell, and a progenitor cell.

104. The eukaryotic cell of claim 99, wherein the mtDNA-encoded peptide is ATPase 6 subunit of F₀F₁-ATP synthase or ND4 subunit of complex I.

105. The eukaryotic cell of claim 99, wherein the organelle-targeting signal is selected from the group consisting of the N-terminal region of human cytochrome c oxidase subunit VIII, the N-terminal region of the P1 isoform of subunit c of human ATP synthase, and the N-terminal region of the aldehyde dehydrogenase targeting sequence.

106. The eukaryotic cell of claim 99, wherein the expression vector transforms the cells by a method selected from the group consisting of electroporation, DEAE Dextran transfection, calcium phosphate transfection, cationic liposome fusion, protoplast fusion, creation of an in vivo electrical field, DNA-coated microprojectile bombardment, injection with a recombinant replication-defective virus, homologous recombination, ex vivo gene therapy, a viral vector, and naked DNA transfer.

107. The eukaryotic cell of claim 99, wherein the expression vector further comprises a nucleic acid sequence encoding a detectable marker.

108. The eukaryotic cell of claim 107, wherein the detectable marker is a FLAG epitope or green fluorescent protein (GFP).

109. The eukaryotic cell of claim 99, wherein the mtDNA-encoded peptide is ATPase 6 subunit of F₀F₁-ATP synthase, and the organelle-targeting signal is the N-terminal region of human cytochrome c oxidase subunit VIII or the N-terminal region of the P1 isoform of subunit c of human ATP synthase.

110. The eukaryotic cell of claim 99, wherein the mtDNA-encoded peptide is ND4 subunit of complex I, and the organelle-targeting signal is the N-terminal region of the P1 isoform of subunit c of human ATP synthase or the N-terminal region of the aldehyde dehydrogenase targeting sequence.

111. The eukaryotic cell of claim 99, wherein the expression vector is selected from the group consisting of a bicistronic vector, a plasmid vector, and an adeno-associated virus (AAV) vector.

112. A clonal cell strain comprising the transformed eukaryotic cell of claim 99.

113. A pharmaceutical composition, comprising:
(a) a non-nuclear nucleic acid sequence encoding a peptide for introduction into an organelle, wherein the nucleic acid sequence is compatible with the universal genetic code;
(b) a nucleic acid sequence encoding an organelle-targeting signal; and
(c) a pharmaceutically-acceptable carrier.

114. The pharmaceutical composition of claim 113, wherein the peptide is a mitochondrial-DNA-encoded (mtDNA-encoded) peptide.

115. The pharmaceutical composition of claim 114, wherein the mtDNA-encoded peptide is ATPase 6 subunit of F₀F₁-ATP synthase or ND4 subunit of complex I.

116. The pharmaceutical composition of claim 113, wherein the organelle-targeting signal is selected from the group consisting of the N-terminal region of human cytochrome c oxidase subunit VIII, the N-terminal region of the P1 isoform of subunit c of human ATP synthase, and the N-terminal region of the aldehyde dehydrogenase targeting sequence.

117. The pharmaceutical composition of claim 113, wherein the peptide is a mitochondrial-DNA-encoded (mtDNA-encoded) peptide, and the organelle-targeting signal is selected from the group consisting of the N-terminal region of human cytochrome c oxidase subunit VIII, the N-terminal region of the P1 isoform of subunit c of human ATP synthase, and the N-terminal region of the aldehyde dehydrogenase targeting sequence.

118. The pharmaceutical composition of claim 117, wherein the mtDNA-encoded peptide is ATPase 6 subunit of F₀F₁-ATP synthase, and the organelle-targeting signal is the N-terminal region of human cytochrome c oxidase subunit VIII or the N-terminal region of the P1 isoform of subunit c of human ATP synthase.

119. The pharmaceutical composition of claim 117, wherein the mtDNA-encoded peptide is ND4 subunit of complex I, and the organelle-targeting signal is the N-terminal region of the P1 isoform of subunit c of human ATP synthase or the N-terminal region of the aldehyde dehydrogenase targeting sequence.
